(19) United States
(12) Patent Application Publication
(10) Pub. No.: US 2007/0022497 A1
(43) Pub. Date: Jan. 25, 2007

(54) Trans-2-enoyl-coa reductase gene of euglena gracilis

(75) Inventors: Petra Cirpus, Mannheim (DE); Oliver Oswald, Ludwigshafen (DE); Jens Lerchi, Svalov (SE); William Frank Martin, Neuss (DE); Meike Hoffmeister, Dusseldorf (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 10/574,902

(22) PCT Filed: Oct. 8, 2004
(86) PCT No.: PCT/EP04/11294

§ 371 Date: Apr. 7, 2006

(51) Int. Cl. A01H 1/00; C07H 21/04; C12N 9/02; C12N 5/04; C12N 15/82

(52) U.S. Cl. 800/281; 435/189; 435/419; 435/468; 536/23.2

(57) ABSTRACT

The invention relates to the identification and use of a nucleic acid sequence SEQ ID NO: 1 from Euglena gracilis that when expressed will increase the total amount of oil (i.e. triacylglycerols, diacylglycerols, monoacylglycerols, phospholipids, waxesters and/or fatty acids) that is produced in transgenic organisms.

### DESCRIPTION

The invention relates to the use of a nucleic acid sequence SEQ ID NO: 1, SEQ ID NO: 4, SEQ ID NO: 6, SEQ ID NO: 8 or SEQ ID NO: 10 that when expressed will increase the total amount of lipids (i.e. triacylglycerols, diacylglycerols, monoacylglycerols, phospholipids, waxesters and/or fatty acids) that is produced in transgenic organisms.

More specifically this invention describes the identification of a nucleic acid sequence SEQ ID NO: 1 from Euglena gracilis encoding a trans-2-enoyl-CoA reductase (TER—E.C.1.3.1.44).

In another embodiment, this invention is directed to a protein comprising an amino acid sequence as set forth in SEQ ID NO: 2, SEQ ID NO: 5, SEQ ID NO: 7, SEQ ID NO: 9 or SEQ ID NO: 11 or a functional fragment, derivative, variant, or ortologue thereof.

The present invention further includes the nucleotide sequence as set forth in SEQ ID NO: 1, SEQ ID NO: 4, SEQ ID NO: 6, SEQ ID NO: 8or SEQ ID NO: 10 as well as portions of the genomic sequence, the cDNA sequence, allelic variants, synthetic variants and mutants thereof. This includes sequences that are to be used as probes, vectors for transformation or cloning intermediates.

SEQ ID NO: 2, SEQ ID NO: 5, SEQ ID NO: 7, SEQ ID NO: 9or SEQ ID NO: 11 are the deduced amino acid sequences of the open reading frames SEQ ID NO: 1, SEQ ID NO: 4, SEQ ID NO: 6, SEQ ID NO: 8 or SEQ ID NO: 10.

Another aspect of the present invention relates to those polypeptides, which have at least 60% identity to SEQ ID NO: 2, SEQ ID NO: 5, SEQ ID NO: 7, SEQ ID NO: 9 or SEQ ID NO: 11.

The invention furthermore relates to expression constructs for expressing a nucleic acid sequence SEQ ID NO: 1, SEQ ID NO: 4, SEQ ID NO: 6, SEQ ID NO: 8 or SEQ ID NO: 10 encoding a TER gene in plants, preferably in plant seeds, transgenic plants expressing a TER gene and to the use of said transgenic plants for the production of food, feed, seed, pharmaceuticals or fine chemicals, in particular for the production of oils.

In oil crops like e.g. rape, sunflower, oil palms etc., the oil (i.e. triacylglycerols) is the most valuable product of the seeds or fruits and other compounds such as starch, protein and fiber is regarded as by-products with less value. Enhancing the quantity of lipids per weight basis at the expense of other compounds in oil crops would therefore increase the value of the crop. If proteins that promote the allocation of reduced carbon into the production of lipids can be up regulated by overexpression, the cells will accumulate more lipids at the expense of other products. This approach could not only be used to increase the lipids content in already high oil producing organisms such as oil crops, they could also lead to significant lipid production in moderate or low oil containing crops such as soy, oat, maize, potato, sugar beats, and turnips as well as in microorganisms.

Increasing the lipid content in plants and, in particular, in plant seeds is of great interest for traditional and modem plant breeding and in particular for plant biotechnology. Owing to the increasing consumption of vegetable oils for nutrition or industrial applications, possibilities of increasing or modifying vegetable oils are increasingly the subject of current research (for example Töpfer et al. (1995) Science 268:681-686). Its aim is in particular increasing the fatty acid content in seed oils.

The fatty acids which can be obtained from the vegetable oils are also of particular interest. They are employed, for example, as bases for plasticizers, lubricants, surfactants, cosmetics and the like and are employed as valuable bases in the food and feed industries. Thus, for example, it is of particular interest to provide rapeseed oils with fatty acids with medium chain length since these are in demand in particular in the production of surfactants. With regard to medical ramifications, the long chain fatty acids (C18 and longer) found in many seed oils have been linked to reductions in hypercholesterolemia and other clinical disorders related to coronary heart disease (Brenner 1976, Adv. Exp. Med. Biol. 83:85-101). Therefore, consumption of a plant having increased levels of these types of fatty acids may reduce the risk of heart disease. Enhanced levels of seed oil content also increase large-scale production of seed oils and thereby reduce the cost of these oils.

The targeted modulation of plant metabolic pathways by recombinant methods allows the modification of the plant metabolism in an advantageous manner which, when using traditional breeding methods, could only be achieved after a complicated procedure or not at all. Thus, unusual fatty acids, for example specific polyunsaturated fatty acids, are only synthesized in certain plants or not at all in plants and can therefore only be produced by expressing the relevant gene in transgenic plants (for example Millar et al. (2000) Trends Plant Sci 5:95-101).

Triacylglycerols, diacylglycerols, monoacylglycerols, phospholipids, waxesters and other lipids are synthesized from fatty acids. Fatty acid biosynthesis and triacylglycerol biosynthesis can be considered as separate biosynthetic pathways owing to the compartmentalization, but as a single biosynthetic pathway in view of the end product. Lipid synthesis can be divided into two part-mechanisms, one which might be termed “prokaryotic” and another which may be termed “eukaryotic” (Browse et al. (1986) Biochemical J 235:25-31; Ohlrogge & Browse (1995) Plant Cell 7:957-970). The prokaryofic mechanism is localized in the plastids and encompasses the biosynthesis of the free fatty acids which are exported into the cytosol, where they enter the eukaryotic mechanism in the form of fatty acid acyl-CoA esters. In this pathway the fatty acids are esterified by glycerol-3-phosphate acyltransferase and lysophosphaudic acid acyltransferase to the sn-1 and sn-2 positions of glycerol-3-phosphate, respectively, to yield phosphatidic acid (PA). The PA is the precursor for other polar and neutral lipids, the latter being formed in the Kennedy pathway (Voelker 1996, Genetic Engineering ed.:Setlow 18:111-113; Shanklin & Cahoon 1998, Annu. Rev. Plant Physiol. Plant Mol. Biol. 49:611-641; Frentzen 1998, Lipids 100:161-166; Millar et al. 2000, Trends Plant Sci. 5:95-101).

The last step in the synthesis of triacylglycerols has been shown to occur by two different enzymatic reactions, an acyl-CoA dependent reaction catalyzed by an acyl-CoA: diacylglycerol acyltransferase (Cases, S. et al., (1998) Proc. Natl. Acad. Sci., USA 95, 13018-13023.; Lardizabal, et al., 2001) and the acyl-CoA independent reaction catalyzed by an phospholipid : diacylglyerol acyltransferase (Dahlqvist, et al., 2000).

In higher plants waxesters are synthesized from the fatty acids exported from the plastid and their derivatives, particular very long chain fatty acids and their derivatives (Dusty Post-Beittenmiller:Biochemistry and molecular biology of wax production in plants. In Annu. Rev. Plant Physiol.Plant Mol. Biol.(1996); Editor Jones, R L., Vol 47, pp 405-430).

In Euglena gracilis waxesters are formed when grown under anaerobic conditions. Transfer of Euglena from aerobic to anaerobic conditions causes rapid formation of waxesters at the expense of the reserve polysaccharide paramylon. This anaerobic formation of waxesters is coupled by a net synthesis of ATP (Inui, H. et al. (1982) FEBS Lett. 150: 89-93) and, as such, the phenomenon is called waxester fermentation.

In Euglena, four systems of fatty acid synthesis have been described: 1) involving multifunctional fatty acid synthetase in the cytosol, 2) acyl-carrier-protein-dependent systems in the chloroplasts and 3) involving a fatty acid synthetase in microsomes (Kitaoka, S.(1989) Enzymes and their functional location. pp. 2-135 in D. E. Buetow, ed. The biology of Euglena, Vol. 4. Subcellular biochemistry and molecular biology. Academic Press, San Diego).

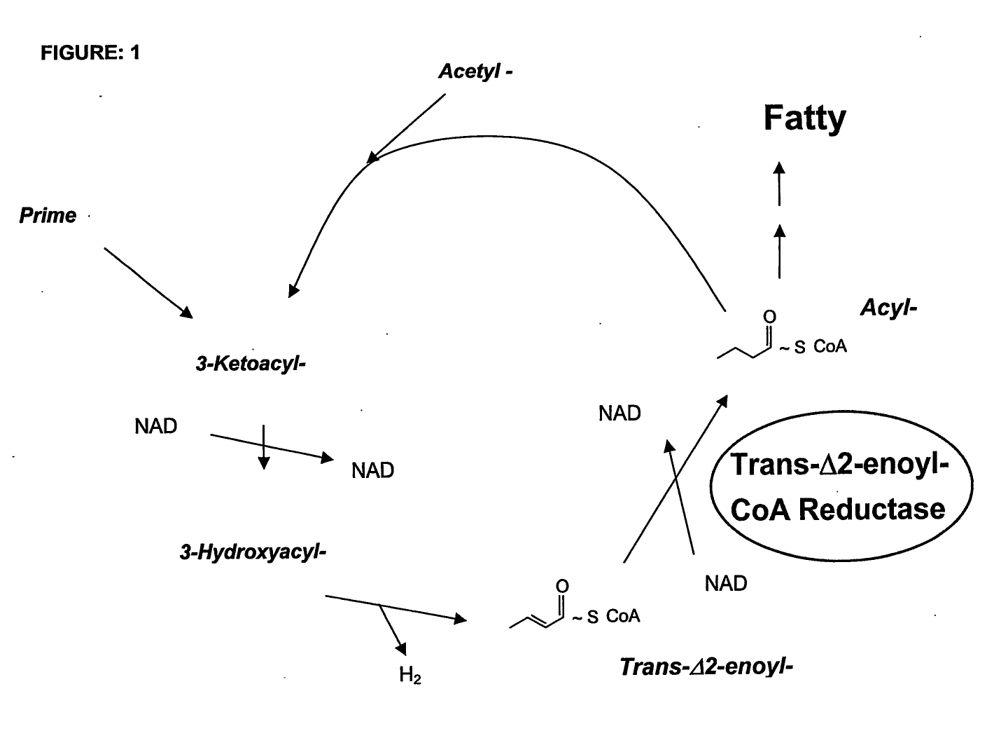

In addition a 4^{th} novel system see FIG. 1, was discovered: In mitochondria of Euglena gracilis, a de novo fatty acid synthesis system was found that is different from the systems in the cytosol (FAS I) and in the chloroplasts (FAS II) (Inui et al. 1982, 1984). The reaction is the reverse of the β-oxidation mechanism of fatty acids with Trans-2-enoyl-CoA reductase (TER) (E.C. 1.3.1.44) replacing acyl-CoA dehydrogenase of the degradative system (Inui, H. et al., Eur. J. Biochem. 142(1984):121-126). Trans-2-enoyl-CoA reductase (TER) is the key enzyme of this fatty acid biosynthetic pathway since it catalyses the last step of the cycle and creates the end product, fatty acyl CoA. Although the enzyme's activity was measured and a partial purification of the protein achieved, protein sequence information for TER was not gained by Inui and co-workers. Without information on the peptide or nucleotide sequence the polypeptide chain, gene and mRNA for this enzyme remained unknown. An enzyme of such function was not described for any other organisms so far.

It is an object of the present invention to provide additional methods for increasing the lipid content in plants.

We have found that this object is achieved by the present invention.

In the present invention we show, that a polynucleotide SEQ ID NO: 1, SEQ ID NO: 4, SEQ ID NO: 6, SEQ ID NO: 8 or SEQ ID NO: 10 present in Euglena gracilis and when over-expressed in plants enhances the amount of triacylglycerols, diacylglycerols, monoacylglycerols, phospholipids, waxesters and/or fatty acids that accumulates in plants.

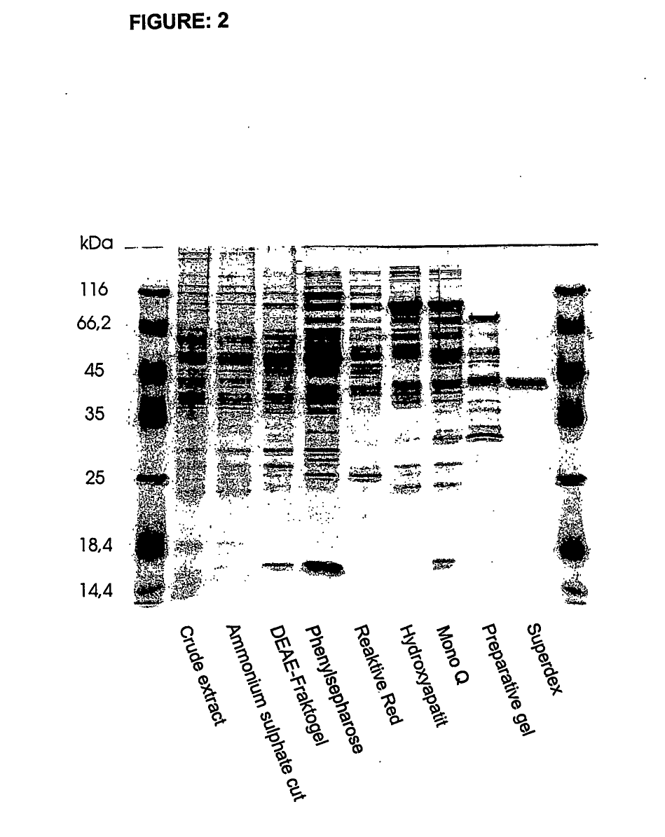

The biochemical purification of trans-2-enoyl-CoA reductase (TER) from Euglena gracilis strain Z was carried out as described in example 1. A series of chromatographic purifications steps was undertaken, see table 4. These included ion exchange chromatography (DEAE-Fraktogel), hydrophobic interactions (phenylsapharose), affinity chromatography (Reaktive Red 120) and hydroxyapatite chromatography. The corresponding purification levels can be seen in table 4. Furthermore, an additional ion exchange chromatography (Mono Q), purification over a preparative gel and a final gel filtration through Superdex 200 completed the purification scheme. This scheme achieved more than 1600 fold purification. When submitted to a SDS-PAGE using standard protocols, the final enzyme preparation showed a major and a thin minor band very closely together at about 44 kDa, see FIG. 2. Enzyme activity was measured as described by Inui and co-workers (Inui et al., 1984) and was associated with the major, upper band.

The major and the minor band were cut from the gel separately and digested with trypsin using standard protocols. The resulting peptides were extracted from the gel and analysed using ESI-Q-TOF MS/MS using standard protocols. Both bands were shown to yield solely identical peptides, confirming the complete purification of the TER as a single subunit enzyme in contrast to the description of Inui and co-workers (Inui, H. et al., (1986) J. Biochem. 100:995-1000).

RNA was isolated from Euglena gracilis cells according to the procedure as described in example 2.

The cDNA library of Euglena gracilis was constructed as described in example 3.

From the cDNA Bank pBluescript phagemides were generated by in-vivo-excision using the ExAssist helper phage for further analysis.

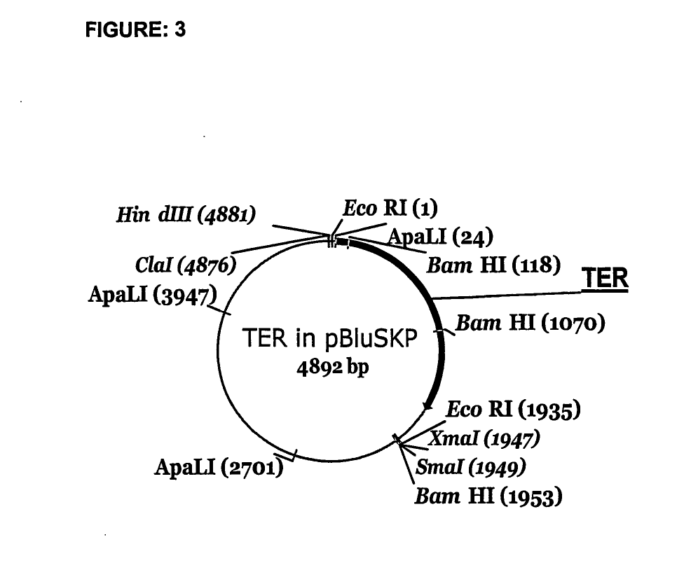

Degenerate primers were designed according to these peptides (see example 4) and used for PCR with cDNA as template. A 837 bp fragment was amplified with the following primers: 5′-GGITGGTAYMYACIGTIGC-3′ (referring to peptide 7) and 5′-GTYTCRTAICCIGCRAARTC-3′ (referring to peptide 9). This fragment was cloned into pBluescript SK+/HincII and sequenced (SEQ ID NO: 3). The translated sequence contained several peptides of the purified protein and therefore the 837 bp fragment was used as hybridisation probe to screen a cDNA library constructed with mRNA from Euglena cells. Screening of 250.000 recombinant phages resulted in six independent clones. cDNA inserts varied between 1600 bp and 1900 bp. Sequencing of all six clones from both ends revealed that all clones represented the same transcript and varied only in length. The longest clone was sequenced completely double-stranded via deletion by exonuclease III. The clone had a length of 1912 bp and encodes an open reading frame of 1620 bp coding for 539 aa (SEQ ID NO: 1 and SEQ ID NO: 2). At both ends it had adaptors consisting of a NotI and EcoRI restriction site and was inserted into the EcoRI site of the vector pBluescript SK+, see FIG. 3.

The clone was liberated from pBluscript SK+ using EcoRI. 5′ overhangs were removed with mung bean nuclease (Roche) using the manufacturer's protocoll and a blunt end ligation using standard protocols performed with the binary vector pSUN 300 or ST593.

The results of the expression studies of Euglena gracilis TER in E. coli—see example 6—can be taken as consideration that the N-terminal part of the cDNA clone may constitute a mitochondrial targeting signal, which has to be cleaved to yield the mature and active TER protein. Nevertheless the possibility that the N-terminal part of the cDNA clone constitutes a transmembran domain can not be excluded. This possible transmembran domain could be lost during biochemical purification of TER from Euglena gracilis (see example 1). If expressed in E. coli this domain may possibly disrubt acticity measurement with the C4-substrat (see example 1) due to incorrect convolution or missing membran-linkage.

A first subject matter of the invention comprises a method of increasing the total lipid content in a plant organism or a tissue, organ, part, cell or propagation material thereof, comprising
a) the transgenic expression of SEQ ID NO: 1, SEQ ID NO: 4, SEQ ID NO: 6, SEQ ID NO: 8 or SEQ ID NO: 10 in said plant organism or in a tissue, organ, part, cell or propagation material thereof, and
b) the selection of plant organisms in which—in contrast to or comparison with the starting organism—the total lipid content in said plant organism or in a tissue, organ, part, cell or propagation material thereof is increased.

Other proteins resulting in the same effect as the protein set forth in SEQ ID NO: 2, SEQ ID NO: 5, SEQ ID NO: 7, SEQ ID NO: 9 or SEQ ID NO: 11 are obtainable from the specific sequences provided herein. Furthermore, it will be apparent that one can obtain natural and synthetic TERs, including those with modified amino acid sequences and starting materials for synthetic-protein modeling from the exemplified TERs and from TERs which are obtained through the use of such exemplified sequences. Modified amino acid sequences include sequences that have been mutated, truncated, increased and the like, whether such sequences were partially or wholly synthesized.

Further, the nucleic acid probes (DNA or RNA) derived from the SEQ ID NO: 1, SEQ ID NO: 4, SEQ ID NO: 6, SEQ ID NO: 8 or SEQ ID NO: 10 of the present invention can be used to screen and recover homologous or related sequences from a variety of plant and microbial sources.

The over-expression of SEQ ID NO: 1, SEQ ID NO: 4, SEQ ID NO: 6, SEQ ID NO: 8 or SEQ ID NO: 10 in yeast or plants will increase the content in total lipids and enhances the amount of triacylglycerols, diacylglycerols, monoacylglycerols, phospholipids, waxesters and/or fatty acids that accumulate compared to wild type yeasts or plants.

The present invention can be characterized by the following aspects:
Example 1 describes the biochemical purification of trans-2-enoyl-CoA reductase (TER) from Euglena gracilis.
Example 2 describes the isolation of total RNA and poly-(A)+ RNA from Euglena gracilis cells.
Example 3 describes the Euglena gracilis cDNA library construction.
Example 4 describes the identification of the TER protein sequence by peptide fingerprinting and subsequent cloning of the corresponding cDNA.
Example 5 describes the triacylglycerol accumulation in yeast cells expressing the TER gene.
Example 6 describes the functional expression of trans-2-enoyl-CoA reductase (TER) in E. coli
Example 7 describes TER constructs for overexpression in Arabidopsis.
Example 8 describes the plasmids for plant transformation.
Example 9 describes the transformation of Arabidopsis.
Example 10 describes the In vitro analysis of the function of the TER gene in transgenic plants.
Example 11 describes the lipid content in transgenic Arabidopsis plants over-expressing the TER gene.
Example 12 describes the amino acid characteristic for trans-enoyl activity based on sequence comparison.

The invention can furthermore be characterized by:

use of the nucleic acid sequence SEQ ID NO: 1, SEQ ID NO: 4, SEQ ID NO: 6, SEQ ID NO: 8 or SEQ ID NO: 10 encoding a protein SEQ ID NO: 2, SEQ ID NO: 5, SEQ ID NO: 7, SEQ ID NO: 9 or SEQ ID NO: 11 that enhances the production of triacylglycerols, diacylglycerols, monoacylglycerols, phospholipids, waxesters and/or fatty acids;

genetic transformation of an oil-producing organism with said sequence in order to be expressed in this organism, resulting in an active protein that increases the lipid content of the organism.

The nucleic acid sequence is derived from the sequence shown in SEQ ID NO: 1 from the Euglena gracilis TER gene (genomic clone or cDNA) or from a nucleic add sequence or cDNA that contains a nucleotide sequence coding for a protein with an amino acid sequence that is 60% or more identical to the amino acid sequence as presented in SEQ ID NO: 2.

The gene product, which we refer to as trans-2-enoyl-CoA reductase (TER) is not itself catalyzing the synthesis of triacylglycerols, diacylglycerols, monoacylglycerols, phospholipids and/or waxesters but its presence elevates the amount of fatty acids synthesized.

The instant invention pertains to a gene construct comprising a said nucleotide sequence SEQ ID NO: 1, SEQ ID NO: 4, SEQ ID NO: 6, SEQ ID NO: 8 or SEQ ID NO: 10 of the instant invention, which is operably linked to a heterologous nucleic acid.

The term “operably linked” means a serial organization e.g. of a promoter, coding sequence, terminator and/or further regulatory elements whereby each element can fulfill its original function during expression of the nucleotide sequence.

Further, a vector comprising the said nucleotide sequence SEQ ID NO: 1, SEQ ID NO: 4, SEQ ID NO: 6, SEQ ID NO: 8 or SEQ ID NO: 10 of the instant invention is contemplated in the instant invention. This includes also an expression vector which can harbor a selectable marker gene and/or nucleotide sequences for the replication in a host cell and/or the integration into the genome of the host cell.

Furthermore, this invention relates to a method for producing a TER encoded by the nucleic acid sequence SEQ ID NO: 1, SEQ ID NO: 4, SEQ ID NO: 6, SEQ ID NO: 8 or SEQ ID NO: 10 in a host cell or progeny thereof including genetically engineered oil seeds, yeast and moulds or any other oil-accumulating organism, via the expression of a construct in the cell. Of particular interest is the expression of the nucleotide sequence of the present invention from transcription initiation regions that are preferentially expressed in plant seed tissues. It is further contemplated that an artificial gene sequence encoding a TER may be synthesized, especially to provide plant-preferred codons. Transgenic cells containing a TER as a result of the expression of a TER encoding sequence are also contemplated within the scope of the invention.

Further, the invention pertains a transgenic cell or organism containing a said nucleotide sequence and/or a said gene construct and/or a said vector. The object of the instant invention is further a transgenic cell or organism which is an eucaryotic cell or organism. Preferably, the transgenic cell or organism is a yeast cell or a plant cell or a plant. The instant invention further pertains said transgenic cell or organism having an increased biosynthetic pathway for the production of substrates for the synthesis of triacylglycerol. A transgenic cell or organism having increased lipid content is also contemplated within the scope of this invention.

Further, the invention pertains a transgenic cell or organism wherein the activity of a TER encoded by the nucleic acid sequence SEQ ID NO: 1, SEQ ID NO: 4, SEQ ID NO: 6, SEQ ID NO: 8 or SEQ ID NO: 10 is increased in said cell or organism. The increased activity of TER is characterized by an alteration in gene expression, catalytic activity and/or regulation of activity of the enzyme. Moreover, a transgenic cell or organism is included in the instant invention, wherein the increased biosynthetic pathway for the production of substrates for the production of triacylglycerol is characterized e.g. by the increased supply of metabolic precursors of the fatty acid biosynthesis.

In a different embodiment, this invention also relates to methods of using a nucleic acid sequence SEQ ID NO: 1, SEQ ID NO: 4, SEQ ID NO: 6, SEQ ID NO: 8 or SEQ ID NO: 10 for increasing the lipid content within the cells of different organisms.

Further, the invention makes possible a process for elevating the production of triacylglycerol, which comprises growing transgenic cells or organisms under conditions whereby the nucleic acid sequence SEQ ID NO: 1, SEQ ID NO: 4, SEQ ID NO: 6, SEQ ID NO: 8 or SEQ ID NO: 10 is expressed in order to produce a TER protein in these cells which results in the enhanced production of triacylglycerols, diacylglycerols, monoacylglycerols, phospholipids, waxesters and/or fatty acids.

Corresponding genes coding for TER can be isolated from other organisms, especially of the phylum Euglenida, the order Euglenales, the genus Euglena and/or the subgenera Euglena, Calliglena or Discoglena. Species belonging to the subgenus Calliglena: e.g. Euglena sanguinea, Euglena mutabilis, Euglena clara, Euglena velata, Euglena agilis, Euglena caudate, Euglena polymorpha, Euglena granulate, Euglena rostrifera, Euglena repulsans, Euglena anabaena and Euglena satelles. Species belonging to the subgenus Euglena: e.g. Euglena viridis, Euglena stellata, Euglena geniculata, Euglena tristella and Euglena chadefaudii. Species belonging to the subgenus Discoglena: e.g. Euglena acus, Euglena texta, Euglena tripteris, Euglena desces, Euglena oxyuris, Euglena spirogyra, Euglena helicoidea, Euglena proxima and Euglena ehrenbergii.

Corresponding genes coding for TER can be isolated from closely related genera like Colacium, Eutreptia, Eutreptiella, Phacus, Lepocinclis, Cryptoglena, Trachelomonas, Strombomonas, Ascoglena, Klebsiella, Astasia, Rhabdomonas, Menoidium, Peranema, Anisonema, Tetreutreptia, Hyalophacus, Khawkinea, Distigma, Cyclidiopsis, Entosiphon, Ploeotia, Gyropaigne, Notoselenus, Petalomonas, Parmidium.

Furthermore the invention pertains transgenic organisms comprising, in their genome or on a plasmid, a nucleic acid sequence SEQ ID NO: 1, SEQ ID NO: 4, SEQ ID NO:

6, SEQ ID NO: 8 or SEQ ID NO: 10 according to the above, transferred by recombinant DNA technology. One important type of transgenic organism covered by this invention are commercially relevant plants in which said nucleic acid sequence preferably would be expressed under the control of a storage organ specific promoter. Alternatively, the nucleic acid sequence could also be expressed under the control of a seed-specific promoter or any other promoter suitable for tissue-specific high-level expression in plants.

The invention also pertains a protein encoded by a DNA molecule according to SEQ ID NO: 1, SEQ ID NO: 4, SEQ ID NO: 6, SEQ ID NO: 8 or SEQ ID NO: 10 or a functional biologically active fragment thereof having TER activity in transgenic organisms. Alternatively, the invention pertains a protein produced in an organism, which has the amino acid sequence set forth in SEQ ID NO: 2, SEQ ID NO: 5, SEQ ID NO: 7, SEQ ID NO: 9 or SEQ ID NO: 11 or an amino acid sequence with at least 60% homology to said amino acid sequence having TER activity.

The protein can be is isolated from Euglena gracilis—as described in example 1—but also from other organisms.

An TER can be recovered from plant material by various methods well known in the art. Organs of plants can be separated mechanically from other tissue or organs prior to isolation of the seed storage compound from the plant organ. Following homogenization of the tissue, cellular debris is removed by centrifugation and the supernatant fraction containing the soluble proteins is retained for further purification of the desired compound. If the product is secreted from cells grown in culture, then the cells are removed from the culture by low-speed centrifugation and the supernatant fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey J. E. & Ollis D. F. 1986, Biochemical Engineering Fundamentals, McGraw-Hill:New York). The purification protocol described herein for purification from Euglena gracilis can be taken as an example and can easily be adapted to other organisms by those skilled in the art.

The identity and purity of the isolated compounds may be assessed by techniques standard in the art. These include high-performance liquid chromatography (HPLC), spectroscopic methods, staining methods, thin layer chromatography, analytical chromatography such as high performance liquid chromatography, NIRS, enzymatic assay, or microbiologically. Such analysis methods are reviewed in: Patek et al. (1994, Appl. Environ. Microbiol. 60:133-140), Malakhova et al. (1996, Biotekhnologiya 11:27-32) and Schmidt et al. (1998, Bioprocess Engineer 19:67-70), Ulmann's Encyclopedia of Industrial Chemistry (1996, Vol. A27, VCH: Weinheim, p. 89-90, p. 521-540, p. 540-547, p. 559-566, 575-581 and p. 581-587) and Michal G. (1999, Biochemical Pathways: An Atlas of Biochemistry and Molecular Biology, John Wiley and Sons; Fallon, A. et al. 1987, Applications of HPLC in Biochemistry in: Laboratory Techniques in Biochemistry and Molecular Biology, vol.17).

The invention additionally pertains the use of a protein according to SEQ ID NO: 2, SEQ ID NO: 5, SEQ ID NO: 7, SEQ ID NO: 9or SEQ ID NO: 11 or derivatives of that protein having TER activity and resulting in increased production of triacylglycerols, diacylglycerols, monoacylglycerols, phospholipids, waxesters and/or fatty acids.

Surprisingly, it has been found that the heterologous expression of the TER gene of Euglena gracilis leads to a significantly increased triacylglycerol, diacylglycerol, monoacylglycerol, phospholipid, waxester and/or fatty acid content ( storage oil) in the seeds of Arabidopsis thaliana as described in example 11. The lipid content was increased by approximately 5%, in one transgenic line even by 10%, compared with wild-type control plants. The over-expression of the nucleic acid sequence encoding a TER protein has no adverse effects on the growth or other properties of the transformed plants.

The method according to the invention can be applied in principle to all plant species, in addition to the species Arabidopsis thaliana, which is employed as model plant. The method according to the invention is preferably applied to lipid crops whose oil content is already naturally high and/or for the industrial production of oils.

Plant organism or tissue, organ, part, cell or propagation material thereof is generally understood as meaning any single- or multi-celled organism or a cell, tissue, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. Also included are mature plants, seeds, shoots and seedlings, and parts, propagation material (for example tubers, seeds or fruits) and cultures derived from them, for example cell cultures or callus cultures.

Plant encompasses all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Anbrrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea and Populus.

Preferred plants are those from the following plant families: Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae.

Preferred monocotyledonous plants are selected in particular from the monocotyledonous crop plants such as, for example, the Gramineae family, such as rice, maize, wheat or other cereal species such as barley, millet and sorghum, rye, triticale or oats, and sugar cane, and all grass species.

The invention is applied very particularly preferably to dicotyledonous plant organisms. Preferred dicotyledonous plants are selected in particular from the dicotyledonous crop plants such as, for example,
Asteraceae such as Heliantus annuus (sunflower), tagetes or calendula and others,
Compositae, especially the genus Lactuca, very particularly the species sativa (lettuce) and others,
Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages;

and the genus Arabidopsis, very particularly the species thaliana, and cress or canola and others,
Cucurbitaceae such as melon, pumpkin/squash or zucchini and others,
Leguminosae, particularly the genus Glycine, very particularly the species max (soybean), soya, and alfalfa, pea, beans or peanut and others,
Rubiaceae, preferably the subclass Lamiidae such as, for example Coffea arabica or Coffea liberica (coffee bush) and others,
Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others,
Sterculiaceae, preferably the subclass Dilleniidae such as, for example, Theobroma cacao (cacao bush) and others,
Theaceae, preferably the subclass Dilleniidae such as, for example, Camellia sinensis or Thea sinensis (tea shrub) and others,
Umbelliferae, particularly the genus Daucus (very particularly the species carota (carrot)) and Apium (very particularly the species graveolens dulce (celeary)) and others;
   and linseed, cotton, hemp, flax, cucumber, spinach, carrot, sugar beet and the various tree, nut and grapevine species, in particular banana and kiwi fruit

Also encompassed are ornamental plants, useful or ornamental trees, flowers, cut flowers, shrubs or turf plants which may be mentioned by way of example but not by limitation are angiosperms, bryophytes such as, for example, Hepaticae (liverworts) and Musci (mosses); pteridophytes such as fems, horsetail and clubmosses; gymnosperms such as conifers, cycades, ginkgo and Gnetatae; algae such as Chlorophyceae, Phaeophpyceae, Rhodophyceae, Myxophyceae, Xanthophyceae, Bacillariophyceae (diatoms) and Euglenophyceae. Plants within the scope of the invention comprise by way of example and not by way of limitation, the families of the Rosaceae such as rose, Ericaceae such as rhododendron and azalea, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geranium, Liliaceae such as dracena, Moraceae such as ficus, Araceae such as cheeseplant and many others.

Furthermore, plant organisms for the purposes of the invention are further organisms capable of being photosynthetically active such as, for example, algae, cyanobacteria and mosses. Preferred algae are green algae such as, for example, algae from the genus Haematococcus, Phaedactylum tricomatum, Volvox or Dunaliella. Synechocystis is particularly preferred.

Most preferred are oil crops. Oil crops are understood as being plants whose lipid content is already naturally high and/or which can be used for the industrial production of oils. These plants can have a high lipid content and/or else a particular fatty acid composition which is of interest industrially. Preferred plants are those with a lipid content of at least 1% by weight. Oil crops encompassed by way of example: Borvago officinalis (borage); Brassica species such as B. campestdis, B. napus, B. rapa (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hidisutfum (American cotton); Gossypium barbadense (Egyptan cotton); Gossypium herbaceum (Asian cotton); Helianthus annuus (sunflower); Linum usitatissimum (linseed or flax); Oenothera biennis (evening primrose); Olea europaea (olive); Oryza sativa (rice); Ricinus communis (castor); Sesamum indicum (sesame); Triticum species (wheat); Zea mays (maize), and various nut species such as, for example, walnut or almond.

“Total lipid content” refers to the sum of all oils, preferably to the sum of the triacylglycerols, diacylglycerols, monoacylglycerols, phospholipids, waxesters and/or fatty acids.

“Lipids” encompasses neutral and/or polar lipids and mixtures of these. Those mentioned in Table 1 may be mentioned by way of example, but not by limitation.

TABLE 1
Classes of plant lipids

| | |
|---|---|
| Neutrale lipids | Triacylglycerol (TAG) |
| | Diacylglycerol (DAG) |
| | Monoacylglycerol (MAG) |
| Polar lipids | Monogalactosyldiacylglycerol (MGDG) |
| | Digalactosyldiacylglycerol (DGDG) |
| | Phosphatidylglycerol (PG) |
| | Phosphatidylcholine (PC) |
| | Phosphatidylethanolamine (PE) |
| | Phosphatidylinositol (PI) |
| | Phosphatidylserine (PS) |
| | Sulfoquinovosyldiacylglycerol |

Neutral lipids preferably refers to triacylglycerols. Both neutral and polar lipids may comprise a wide range of various fatty acids. The fatty acids mentioned in Table 2 may be mentioned by way of example, but not by limitation.

TABLE 2
Overview over various fatty acids (selection)

| Nomenclature¹ | Name |
|---|---|
| 16:0 | Palmitic acid |
| 16:1 | Palmitoleic acid |
| 16:3 | Roughanic acid |
| 18:0 | Stearic acid |
| 18:1 | Oleic acid |
| 18:2 | Linoleic acid |
| 18:3 | Linolenic acid |
| γ-18:3-18:3 | Gamma-linolenic acid* |
| 20:0 | Arachidic acid |
| 22:6 | Docosahexaenoic acid (DHA)* |
| 20:2 | Eicosadienoic acid |
| 20:4 | Arachidonic acid (AA)* |
| 20:5 | Eicosapentaenoic acid (EPA)* |
| 22:1 | Erucic acid |

¹Chain length: number of double bonds
*not naturally occurring in plants

Oils preferably relates to seed oils.

“Increase in” the total lipid content refers to the increased lipid content in a plant or a part, tissue or organ thereof, preferably in the seed organs of the plants. In this context, the lipid content is at least 5%, preferably at least 10%, particularly preferably at least 15%, very particularly preferably at least 20%, most preferably at least 25% increased under otherwise identical conditions in comparison with a starting plant which has not been subjected to the method according to the invention, but is otherwise unmodified. Conditions in this context means all of the conditions which are relevant for germination, culture or growth of the plant, such as soil conditions, climatic conditions, light conditions, fertilization, irrigation, plant protection treatment and the like.

“TER” generally refers to all those proteins which are capable of having the enzymatic activity of a trans-2-enoyl-CoA reductase and resulting if a corresponding nucleic acid SEQ ID NO: 1,SEQ ID NO: 4, SEQ ID NO: 6, SEQ ID NO: 8 or SEQ ID NO: 10 is expressed in an increased lipid content in oil producing organisms, especially microorganisms, yeast, fungi and plants and said proteins are identical to SEQ ID NO: 2, SEQ ID NO: 5, SEQ ID NO: 7, SEQ ID NO: 9 or SEQ ID NO: 11 or have homology to SEQ ID NO: 2, SEQ ID NO: 5, SEQ ID NO: 7, SEQ ID NO: 9 or SEQ ID NO: 11.

TER refers in particular to the polypeptide sequence SEQ ID NO: 2, SEQ ID NO: 5, SEQ ID NO: 7, SEQ ID NO: 9 or SEQ ID NO: 11.

Most preferably, TER refers to the Euglena gracilis protein TER as shown in SEQ ID NO: 2 and functional equivalents or else functionally equivalent portions of the above.

Trans-2-enoly-CoA-reductase can be recovered from different organisms or plant material by various methods well known in the art. Organs of plants can be separated mechanically from other tissue or organs prior to isolation of the seed storage compound from the plant organ. Following homogenization of the tissue, cellular debris is removed by centrifugation and the supernatant fraction containing the soluble proteins is retained for further purification of the desired compound. If the product is secreted from cells grown in culture, then the cells are removed from the culture by low-speed centrifugation and the supernatant fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There is a wide array of purification methods known in the art and the preceding method of purification is not meant to be limiting. Such purification techniques are described, for example, in Bailey J. E. & Ollis D. F. 1986, Biochemical Engineering Fundamentals, McGraw-Hill:New York). The purification protocol described herein for purification from Euglena gracilis can be taken as an example and can easily be adapted to other organisms by those skilled in the art.

“Functional equivalents” refers in particular to natural or artificial mutations of the Euglena gracilis protein TER as shown in SEQ ID NO: 2, SEQ ID NO: 5, SEQ ID NO: 7, SEQ ID NO: 9 or SEQ ID NO: 11 and homologous polypeptides from other organisms belong e.g. to the phylum Euglenida which have the same essential characteristics of TER as defined above. Mutations encompass substitutions, additions, deletions, inversions or insertions of one or more amino acid residues.

The TER to be employed advantageously within the scope of the present invention can be found readily by database searches or by screening gene or cDNA libraries using the polypeptide sequence shown in SEQ ID NO: 2, which is given by way of example, or the nucleic acid sequence as shown in SEQ ID NO: 1, which encodes the latter, as search sequence or probe.

Said functional equivalents preferably have at least 60%, particularly preferably at least 70%, particularly preferably at least 80%, most preferably at least 90% homology with the protein of SEQ ID NO: 2.

Furthermore the nucleic acid sequence SEQ ID NO: 3 can be used in order to identify and clone genes encoding a TER from organisms having at least 60% homology to SEQ ID NO: 3.

The functional equivalent of SEQ ID NO: 2 has an identity of at least 50%, 51%, 52%, 53%, 54%, 55%, 56%, 57% preferably at least 58%, 59%, 60%, 61%, 62%, 63%, 64%, 65%, 66%, 67%, 68%, 69%, and 70% more preferably 71%, 72%, 73%, 74%, 75%, 76%, 77%, 78%, 79%, 80%, 81%, 82%, 83%, 84%, 85% most preferably at least 86% 87%, 88%, 89%, 90%, 91%, 92%, 93%, 94%, 95%, 96%, 97%, 98%, 99% identity with the SEQ ID NO: 2.

Furthermore the following nucleic acid sequences can be used in order to identify and clone genes encoding a TER from different organisms and having at least 40% identity to SEQ ID NO: 2:

Sequences from SwissProt/TREMBL (Code & species):
Q87QB9 Vibrio parahaemolyticus
Q8D8Y6 Vibrio vulnificus
Q8PE66 Xanthomonas campestris (pv. campestris)
Q83EP5 Coxiella bumetii.
Q8PR25 Xanthomonas axonopodis (pv. citri)
Q8EG14 Shewanella oneidensis
Q87CN3 Xylella fastidiosa
Q88E33 Pseudomonas putida
Q8XIP1 Clostridium perfringens
Q8D795 Vibrio vulnificus.
Q93HE4 Streptomyces avermitilis
Q87HT6 Vibrio parahaemolyticus

Sequences from PIR (Code & species):
A83277 Pseudomonas aeruginosa
H82630 Xylella fastidiosa
AD0498 Yersinia pestis
B82164 Vibrio cholerae
B82418 Vibrio cholerae
G96956 Clostridium acetobutylicum

Sequences from Genbank REFSEQ (Code & species):
ZP00116993 Cytophaga hutchinsonii
ZP00064975 Microbulbifer degradans
ZP00033810 Burkholderia fungorum

Table 3 shows the amino acid sequence of TER compared to the amino acid sequence of these homologous nucleic acids and the corresponding conserved amino acid residues. Conserved residues are shaded in grey. The consensus for these residues is given below the sequences: capital letters denote mandatory residues; regular letters give the most prominent amino acid of a mandatory similarity group at this location.

By comparision with functionally characterised sequences putative functional sites of SEQ ID NO: 2 may be assigned to the following stretches of amino acids:
NADH binding site amino acids 190 to196 of SEQ ID NO: 2 (Jörnvall et al. (1995) “Short-chain dehydrogenases/reductases(SDR)”. Biochem. 34: 6003-6013.)
Short chain dehydrogenase/reductase catalytic sites amino acids 231 to 238 and 279 to 285 of SEQ ID NO: 2 (Das et al.( 2000) “Molecular cloning and expression of mammalian peroxisomal trans-2-enoyl-coenzyme A reductase cDNAs.” J. Biol. Chem. 275: 24333-24340).

FAD binding site amino acids 515 to 520 of SEQ ID NO: 2 (Chang & Hammes (1989) “Homology analysis of the protein sequences of fatty acid synthases from chicken liver, rat mammary gland, and yeast.” Proc. Natl. Acad. Sci. USA 86: 8373-8376.)

TABLE 3

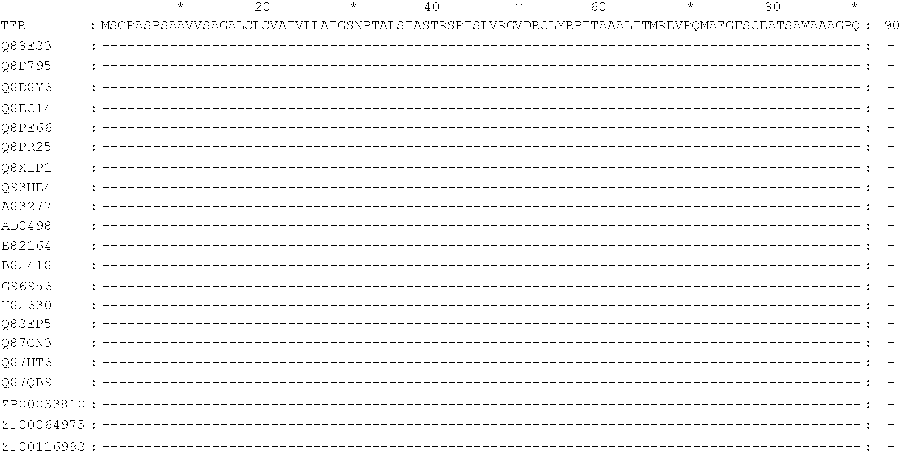

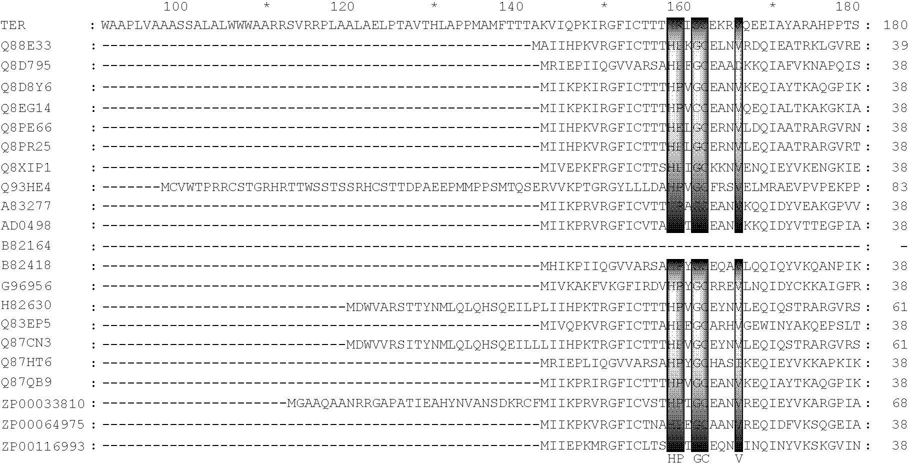

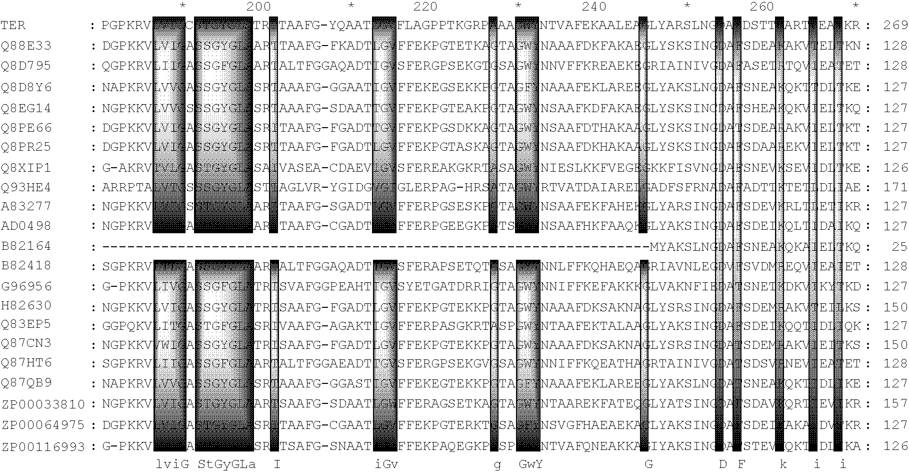

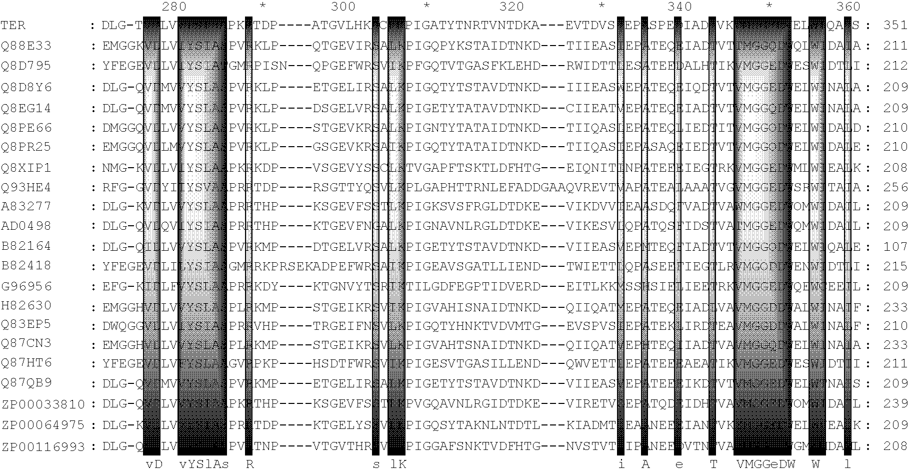

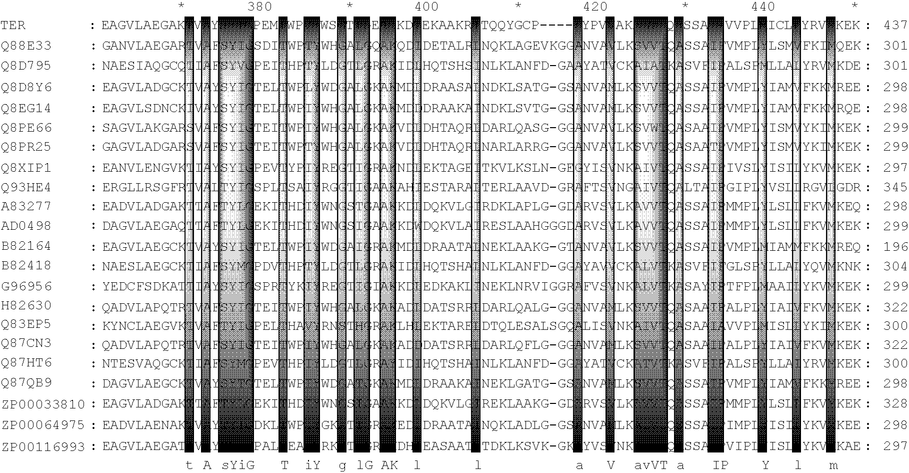

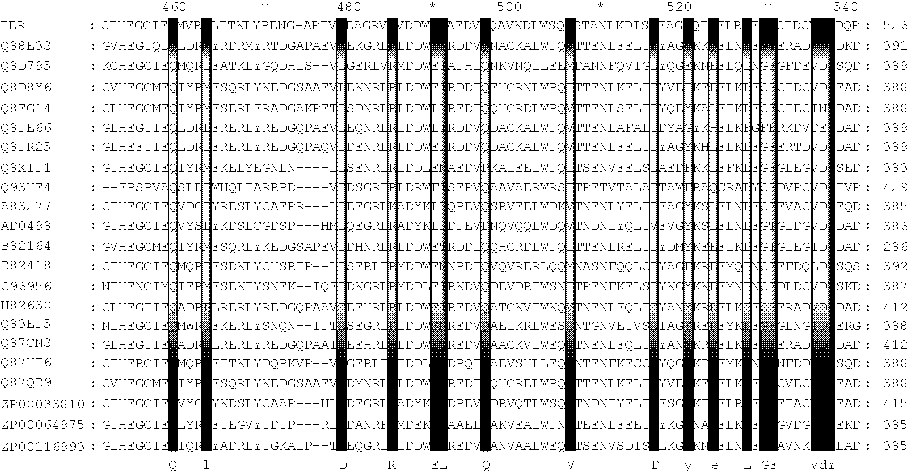

Homology between two polypeptides is understood as meaning the identity of the amino acid sequence over the entire sequence length which is calculated by comparison with the aid of the program algorithm GAP (Wisconsin Package Version 10.0, University of Wisconsin, Genetics Computer Group (GCG), Madison, USA), setting the following parameters:

| | |
|---|---|
| Gap Weight: 8 | Length Weight: 2 |
| Average Match: 2,912 | Average Mismatch: −2,003 |

For example, a sequence with at least 80% homology with the sequence SEQ ID NO: 2 at the protein level is understood as meaning a sequence which, upon comparison with the sequence SEQ ID NO: 2 with the above program algorithm and the above parameter set has at least 80% homology.

Functional equivalents—for example—also encompass those proteins which are encoded by nucleic acid sequences which have at least 60%, particularly preferably at least 70%, particularly preferably at least 80%, most preferably at least 90% homology with the nucleic acid sequence SEQ ID NO: 1.

Homology between two nucleic acid sequences is understood as meaning the identity of the two nucleic acid sequences over the entire sequence length which is calculated by comparison with the aid of the program algorithm GAP (Wisconsin Package Version 10.0, University of Wisconsin, Genetics Computer Group (GCG), Madison, USA), setting the following parameters:

| | |
|---|---|
| Gap Weight: 50 | Length Weight: 3 |
| Average Match: 10 | Average Mismatch: 0 |

For example, a sequence which has at least 80% homology with the sequence SEQ ID NO: 1 at the nucleic acid level is understood as meaning a sequence which, upon comparison with the sequence SEQ ID NO: 1 within the above program algorithm with the above parameter set has a homology of at least 80%.

Functional equivalents also encompass those proteins which are encoded by nucleic acid sequences which hybridize under standard conditions with a nucleic acid sequence described by SEQ ID NO: 1 or a nucleic acid sequence which is complementary thereto or parts of the above and which have the essential characteristics of an TER as characterised by SEQ ID NO: 2.

Natural examples of TERs and the corresponding genes can furthermore readily be found in various organisms whose genomic sequence is unknown, by hybridization techniques in a manner known per se, for example starting from the nucleic acid sequences SEQ ID NO: 1 or SEQ ID NO: 3.

The hybridization may be carried out under moderate (low stringency) or, preferably, under stringent (high stringency) conditions.

Such hybridization conditions are described, inter alia, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

By way of example, the conditions during the washing step may be selected from the range of conditions which is limited by those with low stringency (with 2×SSC at 50° C.) and those with high stringency (with 0.2×SSC at 50° C, preferably at 65° C.) (20×SSC: 0.3 M sodium citrate, 3 M sodium chloride, pH 7.0).

In addition, the temperature may be raised during the washing step from moderate conditions at room temperature, 22° C., to stringent conditions at 65° C.

Both parameters, salt concentration and temperature, may be varied simultaneously and it is also possible to keep one of the two parameters constant and to vary only the other one. It is also possible to use denaturing agents such as, for example, formamide or SDS during hybridization. In the presence of 50% formamide, the hybridization is preferably carried out at 42° C.

Some exemplary conditions for hybridization and washing step are listed below:
(1.) hybridization conditions with, for example
   (i) 4×SSC at 65° C., or
   (ii) 6×SSC at 45° C., or
   (iii) 6×SSC at 68° C., 100 mg/ml denatured fish sperm DNA, or
   (iv) 6×SSC, 0.5% SDS, 100 mg/ml denatured fragmented salmon sperm DNA at 68° C., or
   (v) 6×SSC, 0.5% SDS, 100 mg/ml denatured fragmented salmon sperm DNA, 50% formamide at 42° C., or
   (vi) 50% formamide, 4×SSC at 42° C., or
   (vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
   (viii) 2× or 4×SSC at 50° C. (moderate conditions), or
   (ix) 30 to 40% formamide, 2× or 4×SSC at 42° C. (moderate conditions).
(2.) Washing steps of 10 minutes each with, for example
   (i) 0.015 M NaCl/0.0015 M sodium citrate/0.1% SDS at 50° C., or
   (ii) 0.1×SSC at 65° C., or
   (iii) 0.1×SSC, 0.5% SDS at 68° C., or
   (iv) 0.1×SSC, 0.5% SDS, 50% formamide at 42° C., or
   (v) 0.2×SSC, 0.1% SDS at 42° C., or
   (vi) 2×SSC at 65° C. (moderate conditions).

The invention furthermore relates to transgenic expression constructs which can ensure a transgenic expression of a TER as characterised by SEQ ID NO: 2, SEQ ID NO: 5, SEQ ID NO: 7, SEQ ID NO: 9 or SEQ ID NO: 11 or in a plant organism or a tissue, organ, part, cells or propagation material of said plant organism.

The definition given above applies to a TER, with the transgenic expression of a nucleic acid encoding TER and described by the sequence with the SEQ ID NO: 1 being particularly preferred.

In said transgenic expression constructs, a nucleic acid molecule encoding a TER is preferably in operable linkage with at least one genetic control element (for example a promoter) which ensures expression in a plant organism or a tissue, organ, part, cell or propagation material of same.

Especially preferred are transgenic expression cassettes wherein the nucleic acid sequence encoding a TER is described by
a) a sequence SEQ ID NO: 1, SEQ ID NO: 4, SEQ ID NO: 6, SEQ ID NO: 8 or SEQ ID NO: 10 or
b) a sequence derived from a sequence SEQ ID NO: 1, SEQ ID NO: 4, SEQ ID NO: 6, SEQ ID NO: 8 or SEQ ID NO: 10 or in accordance with the degeneracy of the genetic code
c) a sequence which has at least 60% identity with a sequence SEQ ID NO: 1, SEQ ID NO: 4, SEQ ID NO: 6, SEQ ID NO: 8 or SEQ ID NO: 10.

Operable linkage is understood as meaning, for example, the sequential arrangement of a promoter with the nucleic acid sequence encoding a TER which is to be expressed (for example the sequence as shown in SEQ ID NO: 1) and, if appropriate, further regulatory elements such as, for example, a terminator in such a way that each of the regulatory elements can fulfil its function when the nucleic acid sequence is expressed recombinantly. Direct linkage in the chemical sense is not necessarily required for this purpose. Genetic control sequences such as, for example, enhancer sequences can also exert their function on the target sequence from positions which are further removed or indeed from other DNA molecules. Preferred arrangements are those in which the nucleic acid sequence to be expressed recombinantly is positioned behind the sequence acting as promoter so that the two sequences are linked covalently to each other. The distance between the promoter sequence and the nucleic acid sequence to be expressed recombinantly is preferably less than 200 base pairs, particularly preferably less than 100 base pairs, very particularly preferably less than 50 base pairs.

Operable linkage and a transgenic expression cassette can both be effected by means of conventional recombination and cloning techniques as they are described, for example, in Maniatis T, Fritsch E F and Sambrook J (1989) Molecular Cloning: A Laboratory Manual, Cold Spring Harbor Laboratory, Cold Spring Harbor (N.Y.), in Silhavy T J, Berman M L und Enquist L W (1984) Experiments with Gene Fusions, Cold Spring Harbor Laboratory, Cold Spring Harbor (N.Y.), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or of a signal peptide, may also be positioned between the two sequences. Also, the insertion of sequences may lead to the expression of fusion proteins. Preferably, the expression cassette composed of a promoter linked to a nucleic acid sequence to be expressed can be in a vector-integrated form and can be inserted into a plant genome, for example by transformation.

However, a transgenic expression cassette is also understood as meaning those constructs where the nucleic acid sequence encoding an TER is placed behind an endogenous plant promoter in such a way that the latter brings about the expression of the TER.

Promoters which are preferably introduced into the transgenic expression cassettes are those which are operable in a plant organism or a tissue, organ, part, cell or propagation material of same. Promoters which are operable in plant organisms is understood as meaning any promoter which is capable of governing the expression of genes, in particular foreign genes, in plants or plant parts, plant cells, plant tissues or plant cultures. In this context, expression may be, for example, constitutive, inducible or development-dependent.

The following are preferred:
a) Constitutive promoters
   “Constitutive” promoters refers to those promoters which ensure expression in a large number of, preferably all, tissues over a substantial period of plant development, preferably at all times during plant development (Benfey et al.(1989) EMBO J 8:2195-2202). A plant promoter or promoter originating from a plant virus is especially preferably used. The promoter of the CaMV (cauliflower mosaic virus) 35S transcript (Franck et al. (1980) Cell 21:285-294; Odell et al. (1985) Nature 313:810-812; Shewmaker et al. (1985) Virology 140:281-288; Gardner et al. (1986) Plant Mol Biol 6:221- 228) or the 19S CaMV promoter (U.S. Pat. No. 5,352,605; WO 84/02913; Benfey et al. (1989) EMBO J 8:2195-2202) are especially preferred. Another suitable constitutive promoter is the Rubisco small subunit (SSU) promoter (U.S. Pat. No. 4,962,028), the leguminB promoter (GenBank Acc.

No. X03677), the promoter of the nopalin synthase from Agrobacterium, the TR dual promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits, the promoter of the Arabidopsis thaliana nitrilase-1 gene (GenBank Acc. No.: U38846, nucleotides 3862 to 5325 or else 5342) or the promoter of a proline-rich protein from wheat (WO 91/13991), and further promoters of genes whose constitutive expression in plants is known to the skilled worker. The CaMV 35S promoter and the Arabidopsis thaliana nitrilase-1 promoter are particularly preferred.
b) Tissue-specific promoters
   Furthermore preferred are promoters with specificities for seeds, such as, for example, the phaseolin promoter (U.S. Pat. No. 5,504,200; Bustos M M et al. (1989) Plant Cell 1(9):839-53), the promoter of the 2S albumin gene (Joseffson L G et al. (1987) J Biol Chem 262:12196- 12201), the legumine promoter (Shirsat A et al. (1989) Mol Gen Genet 215(2):326-331), the USP (unknown seed protein) promoter (Bäumlein H et al. (1991) Mol Gen Genet 225(3):459-67), the napin gene promoter (U.S. Pat. No. 5,608,152; Stalberg K et al. (1996) L Planta 199:515-519), the promoter of the sucrose binding proteins (WO 00/26388) or the legumin B4 promoter (LeB4; Bäumlein H et al. (1991) Mol Gen Genet 225: 121-128; Bäumlein et al. (1992) Plant Journal 2(2):233-9; Fiedler U et al. (1995) Biotechnology (NY) 13(10):1090f), the Arabidopsis oleosin promoter (WO 98/45461), and the Brassica Bce4 promoter (WO 91/13980).

Further suitable seed-specific promoters are those of the gene encoding high-molecular weight glutenin (HMWG), gliadin, branching enyzme, ADP glucose pyrophosphorylase or starch synthase. Promoters which are furthermore preferred are those which permit a seed-specific expression in monocots such as maize, barley, wheat, rye, rice and the like. The promoter of the Ipt2 or Ipt1 gene (WO 95/15389, WO 95/23230) or the promoters described in WO 99/16890 (promoters of the hordein gene, the glutelin gene, the oryzin gene, the prolamin gene, the gliadin gene, the glutelin gene, the zein gene, the casirin gene or the secalin gene) can advantageously be employed.
c) Chemically inducible promoters
   The expression cassettes may also contain a chemically inducible promoter (review article: Gatz et al. (1997) Annu Rev Plant Physiol Plant Mol Biol 48:89-108), by means of which the expression of the exogenous gene in the plant can be controlled at a particular point in time. Such promoters such as, for example, the PRP1 promoter (Ward et al. (1993) Plant Mol Biol 22:361-366), a salicylic acid-inducible promoter (WO 95/19443), a benzenesulfonamide-inducible promoter (EP 0 388 186), a tetracyclin-inducible promoter (Gatz et al. (1992) Plant J 2:397-404), an abscisic acid-inducible promoter EP 0 335 528) or an ethanol-cyclohexanone-inducible promoter (WO 93/21334) can likewise be used. Also suitable is the promoter of the glutathione-S transferase isoform II gene (GST-II-27), which can be activated by exogenously applied safeners such as, for example, N,N-diallyl-2,2-dichloroacetamide (WO 93/01294) and which is operable in a large number of tissues of both monocots and dicots.

Particularly preferred are constitutive promoters, very particularly preferred seed-specific promoters, in particular the napin promoter and the USP promoter.

In addition, further promoters which make possible expression in further plant tissues or in other organisms such as, for example, E.coli bacteria, may be linked operably with the nucleic acid sequence to be expressed. Suitable plant promoters are, in principle, all of the above-described promoters.

The nucleic acid sequences present in the transgenic expression cassettes according to the invention or transgenic vectors can be linked operably with further genetic control sequences besides a promoter. The term genetic control sequences is to be understood in the broad sense and refers to all those sequences which have an effect on the establishment or the function of the expression cassette according to the invention. Genetic control sequences modify, for example, transcription and translation in prokaryotic or eukaryotic organisms. The transgenic expression cassettes according to the invention preferably encompass a plant-specific promoter 5′-upstream of the nucleic acid sequence to be expressed recombinantly in each case and, as additional genetic control sequence, a terminator sequence 3′-downstream, and, if appropriate, further customary regulatory elements, in each case linked operably with the nucleic acid sequence to be expressed recombinantly.

Genetic control sequences also encompass further promoters, promoter elements or minimal promoters capable of modifying the expression-controlling properties. Thus, genetic control sequences can, for example, bring about tissue-specific expression which is additionally dependent on certain stress factors. Such elements are, for example, described for water stress, abscisic acid (Lam E and Chua N H, J Biol Chem 1991; 266(26): 17131-17135) and thermal stress (Schoffl F et al. (1989) Mol Gen Genetics 217(2-3):246-53).

Further advantageous control sequences are, for example, in the Gram-positive promoters amy and SPO2, and in the yeast or fungal promoters ADC1, MFa, AC, P-60, CYC1, GAPDH, TEF, rp28, ADH.

In principle all natural promoters with their regulatory sequences like those mentioned above may be used for the method according to the invention. In addition, synthetic promoters may also be used advantageously.

Genetic control sequences further also encompass the 5′-untranslated regions, introns or nonencoding 3′-region of genes, such as, for example, the actin-1 intron, or the Adh1-S intron 1, 2 and 6 (for general reference, see: The Maize Handbook, Chapter 116, Freeling and Walbot, Eds., Springer, N.Y. (1994)). It has been demonstrated that these may play a significant role in regulating gene expression. Thus, it has been demonstrated that 5′-untranslated sequences can enhance the transient expression of heterologous genes. Translation enhancers which may be mentioned by way of example are the tobacco mosaic virus 5′ leader sequence (Gallie et al. (1987) Nucl Acids Res 15:8693-8711) and the like. They may furthermore promote tissue specificity (Rouster J et al. (1998) Plant J 15:435-440).

The transient expression cassette can advantageously contain one or more of what are known as enhancer sequences in operable linkage with the promoter, and these make possible an increased recombinant expression of the nucleic acid sequence. Additional advantageous sequences such as further regulatory elements or terminators may also be inserted at the 3′ end of the nucleic acid sequences to be expressed recombinantly. One or more copies of the nucleic acid sequences to be expressed recombinanly may be present in the gene construct.

Polyadenylation signals which are suitable as control sequences are plant polyadenylation signals, preferably those which correspond essentially to Agrobacterium tumefaciens T-DNA polyadenylation signals, in particular those of gene 3 of the T-DNA (octopine synthase) of the Ti plasmid pTiACHS (Gielen et al. (1984) EMBO J 3:835 et seq.) or functional equivalents thereof. Examples of particularly suitable terminator sequences are the OCS (octopin synthase) terminator and the NOS (nopaline synthase) terminator.

Control sequences are furthermore understood as those which make possible homologous recombination or insertion into the genome of a host organism, or removal from the genome. In the case of homologous recombination, for example, the coding sequence of the specific endogenous gene can be exchanged in a directed fashion for a sequence encoding a dsRNA. Methods such as the cre/lox technology permit the tissue-specific, possibly inducible, removal of the expression cassette from the genome of the host organism (Sauer B (1998) Methods. 14(4):381-92). Here, certain flanking sequences are added to the target gene.(lox sequences), and these make possible removal by means of cre recombinase at a later point in time.

A recombinant expression cassette and the recombinant vectors derived from it may comprise further functional elements. The term functional element is to be understood in the broad sense and refers to all those elements which have an effect on generation, replication or function of the expression cassettes, vectors or transgenic organisms according to the invention. Examples which may be mentioned, but not by way of limitation, are:
a) Selection markers which confer resistance to a metabolism inhibitor such as 2-deoxyglucose-6-phosphate (WO 98/45456), antibiotics or biocides, preferably herbicides, such as, for example, kanamycin, G 418, bleomycin, hygromycin, or phosphinothricin and the like. Particularly preferred selection markers are those which confer resistance to herbicides. The following may be mentioned by way of example: DNA sequences which encode phosphinothricin acetyltransferases (PAT) and which inactivate glutamine synthase inhibitors (bar and pat gene), 5-enolpyruvylshikimate-3-phosphate synthase genes (EPSP synthase genes), which confer resistance to Glyphosate (N-(phosphonomethyl)glycine), the gox gene, which encodes Glyphosate-degrading enzyme (Glyphosate oxidoreductase), the deh gene (encoding a dehalogenase which inactivates dalapon), sulfonylurea- and imidazolinone-inactivating acetolactate synthases, and bxn genes which encode nitrilase enzymes which degrade bromoxynil, the aasa gene, which confers resistance to the antibiotic apectinomycin, the streptomycin phosphotransferase (SPT) gene, which permits resistance to streptomycin, the neomycin phosphotransferase (NPTII) gene, which confers resistance to kanamycin or geneticidin, the hygromycin phosphotransferase (HPT) gene, which confers resistance to hygromycin, the acetolactate synthase gene (ALS), which confers resistance to sulfonylurea herbicides (for example mutated ALS variants with, for example, the S4 and/or Hra mutation).
b) Reporter genes which encode readily quantifiable proteins and which allow the transformation efficacy or the expression site or time to be assessed via their color or enzyme activity. Very particularly preferred in this context are reporter proteins (Schenborn E, Groskreutz D. Mol Biotechnol. 1999; 13(1):2944) such as the “green fluorescent protein” (GFP) (Sheen et al. (1995) Plant Journal 8(5):777-784), chloramphenicol transferase, a luciferase (Ow et al. (1986) Science 234:856-859), the aequorin gene (Prasher et al. (1985) Biochem Biophys Res Commun 126(3):1259-1268), β-galactosidase, with β-glucuronidase being very particularly preferred (Jefferson et al. (1987) EMBO J 6:3901-3907).
c) Replication origins which allow replication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).
d) Elements which are required for agrobacterium-mediated plant transformation such as, for example, the right or left border of the T-DNA, or the vir region.

To select cells which have successfully undergone homologous recombination or else cells which have successfully been transformed, it is generally required additionally to introduce a selectable marker which confers resistance to a biocide (for example a herbicide), a metabolism inhibitor such as 2-deoxyglucose-6-phosphate (WO 98/45456) or an antibiotic to the cells which have successfully undergone recombination. The selection marker permits the selection of the transformed cells from untransformed cells (McCormick et al. (1986) Plant Cell Reports 5:81-84).

In addition, said recombinant expression cassette or vectors may comprise further nucleic acid sequences which do not encode a nucleic acid sequence SEQ ID NO: 1, SEQ ID NO: 4, SEQ ID NO: 6, SEQ ID NO: 8 or SEQ ID NO: 10 and whose recombinant expression leads to a further increase in fatty acid biosynthesis. By way of example, but not by limitation, such a proOIL nucleic acid sequence which is additionally expressed recombinantly can be selected from among nucleic acids encoding acetyl-CoA carboxylase (ACCase), glycerol-3-phosphate acyltransferase (GPAT), lysophosphatidate acyltransferase (LPAT), diacylglycerol acyltransferase (DAGAT) and phospholipid:diacylglycerol acyltransferase (PDAT). Such sequences are known to the skilled worker and are readily accessible from databases or suitable cDNA libraries of the respective plants.

An expression cassette according to the invention can advantageously be introduced into an organism or cells, tissues, organs, parts or seeds thereof (preferably into plants or plant cells, tissues, organs, parts or seeds) by using vectors in which the recombinant expression cassettes are present. The invention therefore furthermore relates to said recombinant vectors which encompass a recombinant expression cassette for a nucleic acid sequence SEQ ID NO: 1, SEQ ID NO: 4, SEQ ID NO: 6, SEQ ID NO: 8 or SEQ ID NO: 10.

For example, vectors may be plasmids, cosmids, phages, viruses or else agrobacteria. The expression cassette can be introduced into the vector (preferably a plasmid vector) via a suitable restriction cleavage site. The resulting vector is first introduced into E.coli. Correctly transformed E.coli are selected, grown, and the recombinant vector is obtained with methods known to the skilled worker. Restriction analysis and sequencing may be used for verifying the cloning step. Preferred vectors are those which make possible stable integration of the expression cassette into the host genome.

The invention furthermore relates to transgenic plant organisms or tissues, organs, parts, cells or propagation material thereof which comprise a SEQ ID NO: 1, SEQ ID NO: 4, SEQ ID NO: 6, SEQ ID NO: 8 or SEQ ID NO: 10 as defined above, a transgenic expression cassette for a SEQ ID NO: 1, SEQ ID NO: 4, SEQ ID NO: 6, SEQ ID NO: 8 or SEQ ID NO: 10 or a transgenic vector encompassing such an expression cassette.

Such a transgenic plant organism is generated, for example, by means of transformation or transfection of the corresponding proteins or nucleic acids. The generation of a transformed organism (or a transformed cell or tissue) requires introducing the DNA in question (for example the expression vector), RNA or protein into the host cell in question. A multiplicity of methods is available for this procedure, which is termed transformation (or transduction or transfection) (Keown et al. (1990) Methods in

Enzymology 185:527-537). Thus, the DNA or RNA can be introduced for example directly by microinjection or by bombardment with DNA-coated microparticles. The cell may also be permeabilized chemically, for example with polyethylene glycol, so that the DNA may reach the cell by diffusion. The DNA can also be introduced by protoplast fusion with other DNA-comprising units such as minicells, cells, lysosomes or liposomes. Electroporation is a further suitable method for introducing DNA; here, the cells are permeabilized reversibly by an electrical pulse. Soaking plant parts in DNA solutions, and pollen or pollen tube transformation, are also possible. Such methods have been described (for example in Bilang et al. (1991) Gene 100:247-250; Scheid et al. (1991) Mol Gen Genet 228:104-112; Guerche et al. (1987) Plant Science 52:111-116; Neuhaus et al. (1987) Theor Appl Genet 75:30-36; Klein et al. (1987) Nature 327:70-73; Howell et al. (1980) Science 208:1265; Horsch et al.(1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.)- Academic Press Inc. (1989)).

For plant transformation binary vectors such as pBinAR can be used (Höfgen & Willmitzer 1990, Plant Sci. 66:221-230). Construction of the binary vectors can be performed by ligation of the cDNA in sense or antisense orientation into the T-DNA. 5-prime to the cDNA a plant promoter activates transcription of the cDNA. A polyadenylation sequence is located 3 prime to the cDNA. Tissue-specific expression can be achieved by using a tissue specific promoter. For example, seed-specific expression can be achieved by cloning the napin or LeB4 or USP promoter 5-prime to the cDNA. Also any other seed specific promoter element can be used. For constitutive expression within the whole plant the CaMV 35S promoter can be used. The expressed protein can be targeted to a cellular compartment using a signal peptide, for example for plastids, mitochondria or endoplasmic reticulum (Kermode 1996, Crit. Rev. Plant Sci. 15:285-423). The signal peptide is cloned 5-prime in frame to the cDNA to achieve subcellular localization of the fusion protein.

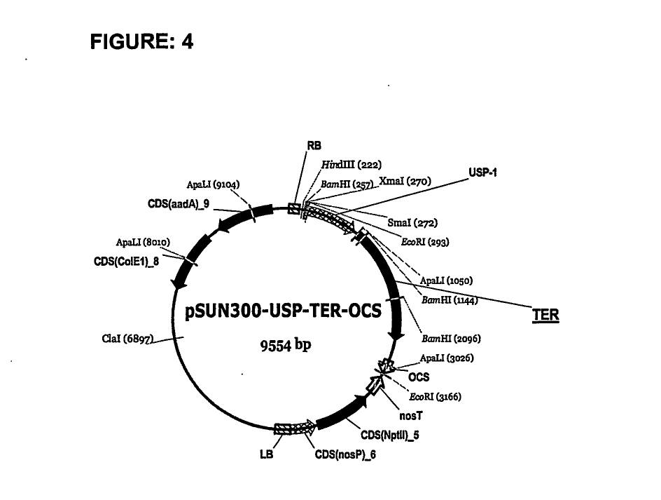

Further examples for plant binary vectors are the pSUN300 vectors into which the TER gene candidates is cloned. These binary vectors contain an antibiotica resistance gene driven under the control of the Nos-promotor and a USP seed-specific promoter in front of the candidate gene with the OCS terminator, see FIG. 4. TER cDNA is cloned into the multiple cloning site of the plant binary vector in sense orientation behind the USP seed-specific promoter. The recombinant vector containing the gene of interest is transformed into Dh5α cells (Invitrogen) using standard conditions. Transformed cells are selected for on LB agar containing 50 μg/ml kanamycin grown overnight at 37° C. Plasmid DNA is extracted using the QIAprep Spin Miniprep Kit (Qiagen) following manufacturer's instructions. Analysis of subsequent clones and restriction mapping is performed according to standard molecular biology techniques (Sambrook et al. 1989, Molecular Cloning, A Laboratory Manual. 2^{nd} Edition. Cold Spring Harbor Laboratory Press. Cold Spring Harbor, N.Y.).

In plants, the methods which have been described for transforming and regenerating plants from plant tissues or plant cells are exploited for transient or stable transformation. Suitable methods are, in particular, protoplast transformation by polyethylene glycol-induced DNA uptake, the biolistic method with the gene gun, what is known as the particle bombardment method, electroporation, the incubation of dry embryos in DNA-containing solution, and microinjection.

In addition to these “direct” transformation techniques, transformation may also be effected by bacterial infection by means of Agrobacterium tumefaciens or Agrobacterium rhizogenes and the transfer of corresponding recombinant Ti plasmids or Ri plasmids by infection with transgenic plant viruses. Agrobacterium-mediated transformation is best suited to cells of dicotyledonous plants. The methods are described, for example, in Horsch R B et al. (1985) Science 225: 1229f.

When agrobacteria are used, the expression cassette is to be integrated into specific plasmids, either into a shuttle vector or into a binary vector. If a Ti or Ri plasmid is to be used for the transformation, at least the right border, but in most cases the right and left border, of the Ti or Ri plasmid T-DNA is linked to the expression cassette to be introduced as flanking region.

Binary vectors are preferably used. Binary vectors are capable of replication both in E.coli and in Agrobacterium. As a rule, they contain a selection marker gene and a linker or polylinker flanked by the right and left T-DNA border sequence. They can be transformed directly into Agrobacterium (Holsters et al. (1978) Mol Gen Genet 163:181-187). The selection marker gene, which is, for example, the nptII gene, which confers resistance to kanamycin, permits a selection of transformed agrobacteria. The Agrobacterium which acts as host organism in this case should already contain a plasmid with the vir region. The latter is required for transferring the T-DNA to the plant cells. An Agrobacterium transformed in this way can be used for transforming plant cells. The use of T-DNA for the transformation of plant cells has been studied intensively and described (EP 120 516; Hoekema, In: The Binary Plant Vector System, Offsetdrukkerij Kanters B. V., Alblasserdam, Chapter V; An et al. (1985) EMBO J 4:277-287). Various binary vectors, some of which are commercially available, such as, for example, pBI101.2 or pBIN19 (Clontech Laboratories, Inc. USA), are known.

Further promoters which are suitable for expression in plants have been described (Rogers et al. (1987) Meth in Enzymol 153:253-277; Schardl et al. (1987) Gene 61:1-11; Berger et al. (1989) Proc Natl Acad Sci USA 86:8402-8406).

Direct transformation techniques are suitable for any organism and cell type. In cases where DNA or RNA are injected or electroporated into plant cells, the plasmid used need not meet any particular requirements. Simple plasmids such as those from the pUC series may be used. If intact plants are to be regenerated from the transformed cells, it is necessary for an additional selectable marker gene to be present on the plasmid.

Stably transformed cells, i.e. those which contain the inserted DNA integrated into the DNA of the host cell, can be selected from untransformed cells when a selectable marker is part of the inserted DNA. By way of example, any gene which is capable of conferring resistance to antibiotics or herbicides (such as kanamycin, G 418, bleomycin, hygromycin or phosphinothricin and the like) is capable of acting as marker (see above). Transformed cells which express such a marker gene are capable of surviving in the presence of concentrations of such an antibiotic or herbicide which kill an untransformed wild type. Examples are mentioned above and preferably comprise the bar gene, which confers resistance to the herbicide phosphinothricin (Rathore K S et al. (1993) Plant Mol Biol 21 (5):871-884), the nptII gene, which confers resistance to kanamycin, the hpt gene, which confers resistance to hygromycin, or the EPSP gene, which confers resistance to the herbicide Glyphosate. The selection marker permits selection of transformed cells from untransformed cells (McCormick et al. (1986) Plant Cell Reports 5:81-84). The plants obtained can be bred and hybridized in the customary manner. Two or more generations should be grown in order to ensure that the genomic integration is stable and hereditary.

The above-described methods are described, for example, in Jenes B et al.(1993) Techniques for Gene Transfer, in: Transgenic Plants, Vol. 1, Engineering and Utilization, edited by S D Kung and R Wu, Academic Press, pp. 128-143, and in Potrykus (1991) Annu Rev Plant Physiol Plant Molec Biol 42:205-225). The construct to be expressed is preferably cloned into a vector which is suitable for transforming Agrobacterium tumefaciens, for example pBin19 (Bevan et al. (1984) Nucl Acids Res 12:8711f).

Agrobacterium mediated plant transformation with the TER nucleic acid described herein can be performed using standard transformation and regeneration techniques (Gelvin, Stanton B. & Schilperoort R. A, Plant Molecular Biology Manual, 2nd ed. Kluwer Academic Publ., Dordrecht 1995 in Sect., Ringbuc Zentrale Signatur:BT11-P; Glick, Bemard R. and Thompson, John E. Methods in Plant Molecular Biology and Biotechnology, S. 360, CRC Press, Boca Raton 1993). For example, Agrobacterium mediated transformation can be performed using the GV3 (pMP90) (Koncz & Schell, 1986, Mol. Gen. Genet. 204:383-396) or LBA4404 (Clontech) Agrobacterium tumefaciens strain. Arabidopsis thaliana can be grown and transformed according to standard conditions (Bechtold 1993, Acad. Sci. Paris. 316:1194-1199; Bent et al. 1994, Science 265:1856-1860). Additionally, rapeseed can be transformed with the TER nucleic acid of the present invention via cotyledon or hypocotyl transformation (Moloney et al. 1989, Plant Cell Report 8:238-242; De Block et al. 1989, Plant Physiol. 91:694-701). Use of antibiotica for Agrobacterium and plant selection depends on the binary vector and the Agrobacterium strain used for transformation. Rapeseed selection is normally performed using kanamycin as selectable plant marker. Additionally, Agrobacterium mediated gene transfer to flax can be performed using, for example, a technique described by Mlynarova et al. (1994, Plant Cell Report 13:282-285).

Transformation of soybean can be performed using for example a technique described in EP 0 424 047, U.S. Pat. No. 5,322,783 (Pioneer Hi-Bred International) or in EP 0 397 687, U.S. Pat. Nos. 5,376,543 or 5,169,770 (University Toledo). Soybean seeds are surface sterilized with 70% ethanol for 4 minutes at room temperature with continuous shaking, followed by 20% (v/v) Clorox supplemented with 0.05% (v/v) Tween for 20 minutes with continuous shaking. Then the seeds are rinsed 4 times with distilled water and placed on moistened sterile filter paper in a Petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile Petri dish and air-dried to a moisture content less than 20% (fresh weight) in a sealed Petri dish until further use.

Agrobacterium tumefaciens culture is prepared from a single colony in LB solid medium plus appropriate antibiotics (e.g. 100 mg/l streptomycin, 50 mg/l kanamycin) followed by growth of the single colony in liquid LB medium to an optical density at 600 nm of 0.8. Then, the bacteria culture is pelleted at 7000 rpm for 7 minutes at room temperature, and re-suspended in MS (Murashige & Skoog 1962, Physiol. Plant. 15:473-497) medium supplemented with 100 mM acetosyringone. Bacteria cultures are incubated in this pre-induction medium for 2 hours at room temperature before use. The axis of soybean zygotic seed embryos at approximately 44% moisture content are imbibed for 2 h at room temperature with the pre-induced Agrobacterium suspension culture. (The imbibition of dry embryos with a culture of Agrobacterium is also applicable to maize embryo axes).

The embryos are removed from the imbibition culture and are transferred to Petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/A carbenicillin or 300 mg/A cefotaxime to kill the agrobacteria.

The method of plant transformation is also applicable to Brassica and other crops. In particular, seeds of canola are surface sterilized with 70% ethanol for 4 minutes at room temperature with continuous shaking, followed by 20% (v/v) Clorox supplemented with 0.05% (v/v) Tween for 20 minutes, at room temperature with continuous shaking. Then, the seeds are rinsed 4 times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use similarly as described in the procedure for soybean embryos.

Once a transformed plant cell has been generated, an intact plant can be obtained using methods known to the skilled worker. For example, callus cultures are used as starting material. The development of shoot and root can be induced in this as yet undifferentiated cell biomass in the known fashion. The plantlets obtained can be planted out and used for breeding.

The skilled worker is familiar with such methods for regenerating plant parts and intact plants from plant cells. Methods which can be used for this purpose are, for example, those described by Fennell et al. (1992) Plant Cell Rep. 11: 567-570; Stoeger et al (1995) Plant Cell Rep. 14:273-278; Jahne et al. (1994) Theor Appl Genet 89:525-533.

“Transgenic”, for example in the case of a TER, refers to a nucleic acid sequence, an expression cassette or a vector comprising said TER nucleic acid sequence or to an organism transformed with said nucleic acid sequence, expression cassette or vector or all those constructs established by recombinant methods in which either
a) the nucleic acid sequence encoding a TER or
b) a genetic control sequence, for example a promoter which is functional in plant organisms, which is linked operably with said nucleic acid sequence under a)
   are not in their natural genetic environment or have been modified by recombinant methods, it being possible for the modification to be, for example, a substitution, addition, deletion, inversion or insertion of one or more nucleotide residues. Natural genetic environment refers to the natural chromosomal locus in the source organism or the presence in a genomic library. In the case of a genomic library, the natural genetic environment of the nucleic acid sequence is preferably retained, at least to some extent. The environment flanks the nucleic acid sequence at least on one side and has a sequence length of at least 50 bp, preferably at least 500 bp, particularly preferably at least 1000 bp, very particularly preferably at least 5000 bp. A naturally occurring expression cassette, for example the naturally occurring combination of the promoter of a gene coding for an TER with the corresponding TER gene, becomes a transgenic expression cassette when the latter is modified by non-natural, synthetic (“artificial”) methods such as, for example, a mutagenization. Such methods are described in U.S. Pat. No. 5,565,350; WO 00/15815; see also above.

Host or starting organisms which are preferred as transgenic organisms are, above all, plants in accordance with the above definition. Included for the purposes of the invention are all genera and species of higher and lower plants of the Plant Kingdom, in particular plants which are used for obtaining oils, such as, for example, oilseed rape, sunflower, sesame, safflower, olive tree, soya, maize, wheat and nut species. Furthermore included are the mature plants, seed, shoots and seedlings, and parts, propagation material and cultures, for example cell cultures, derived therefrom. Mature plants refers to plants at any desired developmental stage beyond the seedling stage. Seedling refers to a young, immature plant at an early developmental stage.

The transgenic organisms can be generated with the above-described methods for the transformation or transfection of organisms.

The invention furthermore relates to the use of the transgenic organisms according to the invention and to the cells, cell cultures, parts—such as, for example, in the case of transgenic plant organisms roots, leaves and the like—and transgenic propagation material such as seeds or fruits which are derived therefrom for the production of food-stuffs or feedstuffs, pharmaceuticals or fine chemicals, in particular oils, fats, fatty acids or derivatives of these.

Besides influencing the lipid content, the transgenic expression of SEQ ID NO: 1, SEQ ID NO: 4, SEQ ID NO: 6, SEQ ID NO: 8 or SEQ ID NO: 10 or derivatives thereof in plants may mediate yet further advantageous effects such as, for example, an increased stress resistance. Such osmotic stress occurs for example in saline soils and water and is an increasing problem in agriculture. Increased stress tolerance makes it possible, for example, to use areas in which conventional arable plants are not capable of thriving for agricultural usage.

The determination of activities and kinetic parameters of enzymes is well established in the art. TER activity can be measured according to Inui et al., (1984), European J. Biochem. 142:121-126.

The activity of a recombinant gene product in the transformed host organism can be measured on the transcriptional or/and on the translational level. A useful method to ascertain the level of transcription of the gene (an indicator of the amount of mRNA available for translation to the gene product) is to perform a Northern blot (for reference see, for example, Ausubel et al. 1988, Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information at least partially demonstrates the degree of transcription of the transformed gene. Total cellular RNA can be prepared from plant cells, tissues or organs by several methods, all well-known in the art, such as that described in Bormann et al. (1992, Mol. Microbiol. 6:317-326).

To assess the presence or relative quantity of protein translated from this mRNA, standard techniques, such as a Western blot, may be employed (see, for example, Ausubel et al. 1988, Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or colorimetric label which may be readily detected. The presence and quantity of label observed indicates the presence and quantity of the desired mutant protein present in the cell.

The effect of the genetic modification in plants on a desired seed storage compound e.g. triacylglycerols, diacylglycerols, monoacylglycerols, phospholipids, waxesters and/or fatty acids can be assessed by growing the modified plant under suitable conditions and analyzing the seeds or any other plant organ for increased production of the desired product. Such analysis techniques are well known to one skilled in the art, and include spectroscopy, thin layer chromatography, staining methods of various kinds, enzymatic and microbiological methods, and analytical chromatography such as high performance liquid chromatography (see, for example, Ullman 1985, Encyclopedia of Industrial Chemistry, vol. A2, pp. 89-90 and 443-613, VCH: Weinheim; Fallon, A. et al. 1987, Applications of HPLC in Biochemistry in: Laboratory Techniques in Biochemistry and Molecular Biology, vol.17; Rehm et al., 1993 Product recovery and purification, Biotechnology, vol. 3, Chapter III, pp. 469-714, VCH: Weinheim; Belter, P. A. et al., 1988 Bioseparations: downstream processing for biotechnology, John Wiley & Sons; Kennedy J. F. & Cabral J. M. S. 1992, Recovery processes for biological materials, John Wiley and Sons; Shaeiwitz J. A. & Henry J. D. 1988, Biochemical separations in: Ulmann's Encyclopedia of Industrial Chemistry, Separation and purification techniques in biotechnology, vol. B3, Chapter 11, pp. 1-27, VCH: Weinheim; and Dechow F. J. 1989).

Besides the above-mentioned methods, plant lipids are extracted from plant material as described by Cahoon et al. (1999, Proc. Natl. Acad. Sci. USA 96, 22:12935-12940) and Browse et al. (1986, Anal. Biochemistry 442:141-145). Qualitative and quantitative lipid or fatty acid analysis is described in Christie, William W., Advances in Lipid Methodology. Ayr/Scotland :Oily Press.—(Oily Press Lipid Library; Christie, William W., Gas Chromatography and Lipids. A Practical Guide—Ayr, Scotland :Oily Press, 1989 Repr. 1992.—IX,307 S. —(Oily Press Lipid Library; and “Progress in Lipid Research, Oxford :Pergamon Press, 1 (1952)—16 (1977) Progress in the Chemistry of Fats and Other Lipids CODEN.

Unequivocal proof of the presence of fatty acid products can be obtained by the analysis of transgenic plants following standard analytical procedures: GC, GC-MS or TLC as variously described by Christie and references therein (1997 in: Advances on Lipid Methodology 4^{th} ed.: Christie, Oily Press, Dundee, pp. 119-169; 1998). Detailed methods are described for leaves by Lemieux et al. (1990, Theor. Appl. Genet. 80:234-240) and for seeds by Focks & Benning (1998, Plant Physiol. 118:91-101).

Positional analysis of the fatty add composition at the C-1, C-2 or C-3 positions of the glycerol backbone is determined by lipase digestion (see, e.g., Siebertz & Heinz 1977, Z. Naturforsch. 32c:193-205, and Christie 1987, Lipid Analysis 2^{nd} Edition, Pergamon Press, Exeter, ISBN 0-08-023791-6).

A typical way to gather information regarding the influence of increased or decreased protein activities on lipid and sugar biosynthetic pathways is for example via analyzing the carbon fluxes by labeling studies with leaves or seeds using ¹⁴C-acetate or ¹⁴C-pyruvate (see, e.g. Focks & Benning 1998, Plant Physiol. 118:91-101; Ecdeston & Ohirogge 1998, Plant Cell 10:613-621). The distribution of carbon-14 into lipids and aqueous soluble components can be determined by liquid scintillation counting after the respective separation (for example on TLC plates) including standards like ¹⁴C-sucrose and ¹⁴C-malate (Eccleston & Ohlrogge 1998, Plant Cell 10:613-621).

Material to be analyzed can be disintegrated via sonification, glass milling, liquid nitrogen and grinding or via other applicable methods. The material has to be centrifuged after disintegration. The sediment is re-suspended in distilled water, heated for 10 minutes at 100° C., cooled on ice and centrifuged again followed by extraction in 0.5 M sulfuric acid in methanol containing 2% dimethoxypropane for 1 hour at 90° C. leading to hydrolyzed oil and lipid compounds resulting in transmethylated lipids. These fatty acid methyl esters are extracted in petrolether and finally subjected to GC analysis using a capillary column (Chrompack, WCOT Fused Silica, CP-Wax-52 CB, 25 m, 0.32 mm) at a temperature gradient between 170° C. and 240° C. for 20 minutes and 5 min. at 240° C. The identity of resulting fatty acid methylesters is defined by the use of standards available form commercial sources (i.e., Sigma).

In case of fatty acids where standards are not available, molecule identity is shown via derivatization and subsequent GC-MS analysis. For example, the localization of triple bond fatty acids is shown via GC-MS after derivatization via 4,4-Dimethoxy-oxazolin-Derivaten (Christie, Oily Press, Dundee, 1998).

For example, yeast expression vectors comprising the nucleic acids disclosed herein, or fragments thereof, can be constructed and transformed into Saccharomyces cerevisiae using standard protocols. The resulting transgenic cells can then be assayed for alterations in sugar, oil, lipid or fatty acid contents.

Similarly, plant expression vectors comprising the nucleic acid SEQ ID NO: 1, SEQ ID NO: 4, SEQ ID NO: 6, SEQ ID NO: 8 or SEQ ID NO: 10 disclosed herein, or fragments thereof, can be constructed and transformed into an appropriate plant cell such as Arabidopsis, soybean, rape, maize, wheat, Medicago truncatula, etc., using standard protocols. The resulting transgenic cells and/or plants derived therefrom can then be assayed for alterations in oil, lipid or fatty acid contents.

Additionally, the sequence SEQ ID NO: 1, SEQ ID NO: 4, SEQ ID NO: 6, SEQ ID NO: 8 or SEQ ID NO: 10 disclosed herein, or fragments thereof, can be used to generate knockout mutations in the genomes of various organisms, such as bacteria, mammalian cells, yeast cells, and plant cells (Girke at al. 1998, Plant J. 15:39-48). The resultant knockout cells can then be evaluated for their composition and content in seed storage compounds, and the effect on the phenotype and/or genotype of the mutation. For other methods of gene inactivation include U.S. Pat. No. 6,004,804 “Non-Chimeric Mutational Vectors” and Puttaraju et al. (1999, “Spliceosome-mediated RNA Frans-splicing as a tool for gene therapy” Nature Biotech. 17:246-252).

The invention now having been generally described will be more readily understood by reference to the following examples, which are included for the purpose of illustration only, and are not intended to limit scope of the present invention.

### EXAMPLES

General Methods

General Cloning Processes:

Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989, Cold Spring Harbor Laboratory Press: ISBN 087969-309-6) or Kaiser, Michaelis and Mitchell (1994, “Methods in Yeast Genetics”, Cold Spring Harbor Laboratory Press: ISBN 0-87969-451-3).

Chemicals:

The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H20 in the following text, from a Milli-Q water system water purification plant (Millipore, Eschborn). Restriction endonucleases, DNA-modifying enzymes and molecular biology kits were obtained from the companies AGS (Heidelberg), Amersham (Braunschweig), Biometra (Göttingen), Boehringer (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, Netherlands). They were used, if not mentioned otherwise, according to the manufacturer's instructions.

Plant Growth:

Arabidopsis thaliana

Plants were either grown on Murashige-Skoog medium as described in Ogas et al. (1997, Science 277:91-94; 1999, Proc. Natl. Acad. Sci. USA 96:13839-13844) or on soil under standard conditions as described in Focks & Benning (1998, Plant Physiol. 118:91-101).

### Example 1

Biochemical purification of trans-2-enoyl-CoA reductase (TER) from Euglena gracilis

One kilogram of Euglena gracilis strain Z cells grown under aerobic conditions for one week was used as starting material.

Euglena cultures were performed in a BIOSTAT B 10L fermenter (Braun Biotech). The culturing conditions were as follows: culturing volume of 7 liters, light intensity of 5000 Ix continuously, temperature of 28° C., stirring at 200 rpm. A defined medium as described by Ogbonna, J. C. et al. (1981) J. Appl. Phycol. 10:67-74 and Yamane, Y et al. (2001) Biotech. Lett. 23:1223-1228 was modified and used. One liter medium was composed of 12 g glucose; 0,8 g KH₂PO₄; 1,5 g (NH₄)₂SO₄; 0,5 g MgSO₄x7H₂O; 0,2 g CaCO₃; 0,0144 g H₃BO₃; 2,5 mg vitamin B₁; 20 μg vitamin B₁₂; 1 ml trace element solution; 1 ml Fe-solution. The trace element solution was composed of 4,4 g ZnSO₄x7H₂O; 1,16 g MnSO₄xH₂O; 0,3 g Na₂MoO₄x2H₂O; 0,32 g CuSO₄x5H₂O; 0,38 g CoSO₄x5H₂O per 100 ml of destined water and the Fe-solution consisted of 1,14 g (NH₄)₂SO₄Fe(SO₄)₂x6H₂O and 1 g EDTA per 100 ml of destined water. The pH of the medium was kept at 2,8 during the cultivation and the cultures were fumigated with 2 liters/min air.

After the culture harvest using standard techniques the cells were disrupted using a French Press. A 30% ammonium sulphate cut was used to remove cell debris. After dialysis, a series of chromatographic purifications steps was undertaken, see table 4. These included ion exchange chromatography (DEAE-Fraktogel), hydrophobic interactions (phenylsapharose), affinity chromatography (Reaktive Red 120) and hydroxyapatite chromatography. The corresponding purification levels can be seen in table 4. Furthermore, an additional ion exchange chromatography (Mono Q), purification over a preparative gel and a final gel filtration through Superdex 200 completed the purification scheme. This scheme achieved more than 1600 fold purification.

The details of the purification procedure were as follows: all chromatographic steps were carried out using a FPLC system (Amersham Biosciences). All columns were packed according to the manufactors instructions or prepacked columns were used. All buffers were filtrated through 0,45 μm nitrocellulose filters (Sartorius, Göttingen) and degassed. After French Press disruption and 30% ammonium sulphate cut the first chromatographic step was carried out with DEAE-Fraktogel EMD 650 S (Merck). 10 runs on a XK 26 column (Amersham Biosciences) with the dimensions of 2,6×12 cm were performed and elution of proteins was achieved with a linear gradient from 0 to 1 M KCl in 25 mM potassiumphosphate buffer pH 6,8 with 1 mM EDTA, 1 mM DTT and 1 μM FAD. Active fractions were pooled and supplied in 4 runs to Phenyl Sepharose 6 Fast Flow low sub (Amersham Biosciences) in a XK 26 column (2,6×14 cm). Elution was performed with a linear gradient from 1 M NH₄(SO₄)₂ descending to 0 M NH₄(SO₄)₂ in 10 mM Tris-HCl pH 8,0 with 1 mM EDTA, 1 mM DTT and 1 μM FAD. Active fractions were pooled and supplied in 5 runs to Reaktive Red 120 (Sigma) in a XK 16 column (1,6×9 cm). Elution was performed with a linear gradient from 0 to 1 M KCl in 25 mM potassiumphosphate buffer pH 6,8 with 1 mM EDTA, 1 mM DTT and 1 μM FAD. Active fractions were pooled again and supplied in 8 runs to Hydroxyapatit matrix using Eco-Pac CHT II Cartridge (Bio-Rad, Muinchen). Elution was performed with a linear gradient from 10 mM potassiumphosphate buffer pH 6,8 with 1 mM DTT and 1 μM FAD to 500 mM potassiumphosphate buffer pH 6,8 with 1 mM DTT and 1 μM FAD. Active fractions were pooled and supplied in 3 runs to Mono Q HR 5/5 column (Amersham Biosciences). Elution was performed with a linear gradient from 0 to 1 M KCl in 10 mM Tris-HCl pH 8,0 with 1 mM EDTA, 1 mM DTT and 1 μM FAD.

Active fractions were pooled and supplied in 6 runs to a 6% continous native polyacrylamid gel in a Mini Prep Cell (Bio-Rad) according to the manufactors instructions. Active fractions were pooled and supplied in 3 runs to a Superdex 200 HR 10/30 column (Amersham Biosciences). Elution was performed with 10 mM Tris-HCl pH 8,0 containing 150 mM NaCl, 1 mM DTT, 1 mM EDTA and 1 μM FAD. Active fractions were submitted to a SDS-PAGE using standard protocols. The final enzyme preparation showed a major and a thin minor band very closely together at about 44 kDa, see FIG. 2.

When submitted to a SDS-PAGE gel (12%) using standard protocols, the final enzyme preparation showed a major and a thin minor band very closely together at about 44 kDa.

Enzyme activity was measured as described by Inui, H. et al. (1984) Eur. J. Biochem. 142: 121-126 with the following modifications. The assay mixture contained 100 mM potassiumphosphate buffer pH 6,2; 0,75 mM Crotonyl-CoA (Sigma); 0,4 mM NADH and 2 μM FAD and enzyme. The assay mixture without substrate was preincubated for 10 min at 30° C. The reaction was also performed at 30° C. and started with the addition of substrate. The activity was determined by the decrease of absorbance at 340 nm. The final assay volume was 1 ml (Ultrospec 2000 Spectrophotometer, Amersham Biosciences) or 200 μl (GENios microplate reader, Tecan Instruments, Maennedorf, Switzerland).

FIG. 2 shows the protein pattern at different purification steps of TER in a SDS-PAGE gel (12%). Enzyme activity was associated with the major, upper band.

TABLE 4
Purification of trans-2-enoyl-CoA reductase
Starting with 1000 g Euglena cells

| | Total activity | Total protein | Specific activity | | yield |
|---|---|---|---|---|---|
| Purification step | (nmols) | (mg) | (nmols/mg/min) | Purification factor | (%) |
| Crude extract | 233569 | 40977 | 5.7 | | |
| Extract after | 57277 | 24903 | 2.3 | | 60.8 |
| ammonium sulphate | | | | | |
| cut & dialysis | | | | | |
| DEAE-Fraktogel | 34473 | 4536 | 7.6 | 3 | 11.1 |
| Phenylsepharose | 14666 | 1164 | 12.6 | 6 | 2.8 |
| Reaktive Red | 14258 | 178 | 80.1 | 35 | 0.43 |
| Hydroxyapatit | 13375 | 58 | 230.6 | 100 | 0.14 |
| Mono Q | 2801 | 8.4 | 333.5 | 145 | 0.02 |
| Preparative gel | 587 | 0.33 | 1778.7 | 773 | 0.0008 |
| Superdex | 559 | 0.144 | 3879.2 | 1687 | 0.0004 |

### Example 2

Isolation of Total RNA and poly-(A)+ RNA from Euglena gracilis cells

For the investigation of transcripts, both total RNA and poly-(A)+ RNA were isolated. RNA is isolated from Euglena gracilis cells according to the following procedure:

The details for the isolation of total DNA relate to the working up of five gram fresh weight of Euglena. The material was triturated under liquid nitrogen in a mortar to give a fine powder and covered with 20 ml Resuspension buffer (50 mM Tris-HCl pH 8,0; 100 mM NaCl; 10 mM EDTA; 30 mM 2-mercaptoethanol; 2% (w/v) SDS; 4 M Guanidinumthiocyanate; 5% (w/v) Polyclar). The homogenate was transferred to a conical tube and extracted by shaking with the same volume of phenol/chloroform/isoamyl alcohol. For phase separation, centrifugation was carried out at 5000g and RT for 5 min. Nucleic acids were precipitated at −20° C. for 60 min using ice-cold isopropanol and then sedimented at 4° C. and 6000 g for 20 min and resuspended in 5 ml of TE buffer which contained 10 μg/ml Proteinase K. Precipitation of RNA was carried out with 1,25 ml 10 M LiCl at −20° C. overnight, followed by sedimentation at 4° C. and 10000 g for 30 min. The RNA pellet was resuspended in 5 ml DEPC-treated water and for further purification precipitated again with ethanol for 2 h at −20° C. Final sedimentation of RNA was achieved by centrifugation at 4° C. and 10000 g for 20 min. The total RNA was diluted in 2 ml TE and the concentration was determined.

The mRNA is prepared from total RNA, using the Amersham Biosciences mRNA purification kit, which utilizes oligo(dT)-cellulose columns.

### Example 3

cDNA Library Construction

For cDNA library construction, first strand synthesis was achieved using Moloney Murine Leukemia Virus reverse transcriptase (Amersham Biosciences, Freiburg, Germany) and oligo-d(T)-primers, second strand synthesis by incubation with DNA polymerase I, Klenow enzyme and RNAseH digestion at 12° C. (30 min) and 22° C. (1 h). The reaction was stopped by incubation at 65° C. (10 min) and subsequently transferred to ice. Nucleotides were removed by phenol/chloroform extraction and Sepharose CL-4B. EcoRI/NotI adapters (Amersham Biosciences, Freiburg) were ligated to the cDNA ends by T4DNA-ligase (Amersham Biocsciences, 12° C., overnight) and phosphorylated by incubation with polynucleotide kinase (Amersham Biosciences, 37° C., 30 min). Excessive adaptors were removed by Sepharose CL-4Bc.DNA molecules were ligated to vector arms and packed into lambda ZAPII phages using the GigapackIII Gold Kit (Stratagene, Amsterdam, Netherlands) using material and following the instructions of the manufacturer.

From the cDNA Bank pBluescript phagemides can be generated by in-vivo-excision using the ExAssist helper phage ( Stratagene, Netherlands) for further analysis.

### Example 4

Identification of TER protein sequence by peptide fingerprinting and subsequent cloning of the corresponding cDNA

SDS-PAGE of the biochemically purified TER resulted in a double band of 44 kDa. The major and the minor band were cut from the gel separately and digested with trypsin using standard protocols. The resulting peptides were extracted from the gel and analysed using ESI-Q-TOF MS/MS using standard protocols. Both bands were shown to yield solely identical peptides, confirming the complete purification of the TER as a single subunit enzyme in contrast to the description of Inui and co-workers (Inui et al. (1986) J. Biochem. 100: 995-1000). These were the peptides identified with ESI-Q-TOF MS/MS:
peptide 1 ACLKPLGATYTNR
peptide 2 AALEAGLYAR
peptide 3 VLVLGCSTGYGLSTR
peptide 4 TDPAT
peptide 5 SLDGDAFDSTTK
peptide 6 DLWSQVNTANLK
peptide 7 AGWYNTVAFEK
peptide 8 RVQEELAYAR
peptide 9 DLSDFAGYQTEFLR
peptide 10 LYPGDGSPLVDEAGR
peptide 11 LTQQYGCPAYPWAK
peptide 12 VDDWEMAEDVQQAVK
peptide 13 STGYG(AMVR/LSEK)
peptide 14 AHPPTSPGPK
peptide 15 ALSEAGVLAEQK
peptide 16 ((GT)/(AS))HEGCLEQMVR
peptide 17 LYPENGAPLVDEQR

Degenerate primers were designed according to these peptides and used for PCR with cDNA as template. Due to the high GC-content of E. gracilis an initial denaturation of 98° C. for 10 min was accomplished prior to PCR. PCR conditions were as follows: 30 cycles with 94° C. for 30 sec; 50° C. for 30 sec and 72° C. for 90 sec; final extension at 72° C. for 5 min. A 837 bp fragment was amplified with the following primers: 5′-GGITGGTAYAAYACIGTIGC-3′ (referring to peptide 7) and 5′-GTYTCRTAICCIGCRAARTC-3′ (referring to peptide 9). This fragment was cloned into pBluescript SK+/HincII and sequenced (SEQ ID NO: 3). The translated sequence contained several peptides of the purified protein and therefore the 837 bp fragment was used as hybridisation probe to screen a cDNA library constructed with mRNA from aerobically grown Euglena cells as described in example 2 and 3. Screening of 250.000 recombinant phages resulted in six independent clones. cDNA inserts varied between 1600 bp and 1900 bp. Sequencing of all six clones from both ends revealed that all clones represented the same transcript and varied only in length. The longest clone was sequenced completely double-stranded via deletion by exonuclease III. The clone had a length of 1912 bp and encodes an open reading frame of 1620 bp coding for 539 aa (SEQ ID NO: 1 and SEQ ID NO: 2). At both ends it had adaptors consisting of a NotI and EcoRI restriction site and was inserted into the EcoRI site of the vector pBluescript SKP. FIG. 3 shows the map of the TER clone in the vector pBluescript SKP.

### Example 5

Triacylglycerol accumulation in yeast cells expressing the TER gene The TER gene can be excised from the cloning vector pBluescript SK+ by EcoRI digestion and cloned into the EcoRI behind the strong inducible GAL1 promotor in the multicopy plasmid pYES2 (Invitrogen), thus generating the plasmid pYTER. The wild type yeast strain By4742 (MATα his3Δ1, leu2 Δ0, lys2 Δ0, ura3 Δ0), transformed with the pYTER is cultivated at 30° C. on a rotary shaker in synthetic medium (Sherman, F. et al., (1986) Laboratory Course Manual for Methods in Yeast Genetics, Cold Spring Harbor Lab. Press, Plainview, N.Y.) lacking uracil and supplemented with 2% (vol/vol) glycerol and 2% (vol/vol) ethanol. The GAL1 promoter is induced after 24 hours of growth by the addition of 2% (wt/vol) final concentration of galactose. Cells are harvested after an additional 24 or 28 hours of growth. Wild type cells By4742, transformed with the empty vector (pYES2) and cultivated under identical conditions, are used as a control.

In order to quantify the total lipid content, 3×5 ml aliquots from yeast cultures are harvested by centrifugation, and the resulting pellets are washed with distilled water and lyophilised. The weight of the dried cells is determined, and the fatty acid content is quantified by conventional gas-liquid chromatography (GLC) analyses after conversion to methyl esters (Dahlqvist et al. (2000) Proc. Natl. Acad. Sci. USA 97, 6487-492).

The lipid content is calculated as nmol fatty acids per mg dry weight. The lipid composition of the yeast is determined in cells harvested from 35-ml liquid cultures. The harvested yeast cells are re-suspended in 15 ml glass tubes in water to a final volume of 0.6 ml, to which 3.75 ml chloroform: methanol (1:2), 50 μl acetic acid, and 2 ml of glass beads (0.45 to 0.50 mm) are added. The yeast cells are disrupted by vigorous agitation (5×1 min) and the lipids are extracted into chloroform according to standard method (Bligh, E. G. and Dyer, W. J., Can. J. Biochem. Physiol. 37(1959), 911-917). The collected lipid fraction is divided in two parts and separated by TLC on Silica Gel 60 plates (Merck) in hexane/diethyl ether/acetic acid (70:30:1) for the quantification of neutral lipids, i.e. unesterified fatty acids (FA), diacylglycerols (DAG), triacylglycerols (TAG), and steryl esters (SE), and in chloroform/methanol/acetic acid: water (85:15:10:3.5) for the quantification of the major polar lipids, i.e. phosphatidylinositol (PI), phosphatidylserine (PS), phosphatidylcholine (PC), and phosphatidylethanolamine (PE). The lipid areas are located by brief exposure to 12 vapors and identified by means of appropriate standards. The different lipid classes are excised from the plates and fatty acid methyl esters are prepared by heating the excised material at 85° C. for 60 min in 2% (vol/vol) sulfuric acid in dry methanol. The methyl esters are extracted and quantified by conventional gas-liquid chromatography (GLC) analyses as described in Dahlqvist et al., 2000.

### Example 6

Functional expression of trans-2-enoyl-CoA reductase (TER) in E.coli

Two different parts of the TER cDNA clone were choosen for heterologous expression in E. coli. The first construct (ter1) encompassed the complete open reading frame (see SEQ ID NO: 1). The second construct (ter2) comprised part of the open reading frame beginning with aa 136. ter2 has a length of 1215 bp and a calculated molecular mass of 45 kDa that is equivalent to the molecular mass of purified TER protein from Euglena gracilis (see FIG. 2). The constructs ter1 and ter2 were amplified from Euglena gracilis cDNA using the following primers:

| | |
|---|---|
| TER1Ndefor | 5′-TAT ACA TAT GTC GTG CCC CGC CTC GCC GTC TG-3′ |
| | Nde I |
| TER1Bglfor | 5′-TAT AGA TCT TAT GTC GTG CCC CGC CTC GCC GTC TG-3′ |
| | Bgl II |
| TER2Ndefor | 5′-TAT ACA TAT GTT CAC CAC CAC AGC GAA GGT CAT CC-3′ |
| | Nde I |
| TERXhorev | 5′-TAT CTC GAG CTA CTG CTG GGC AGC ACT GG-3′ |
| | Xho I |

ter2 was inserted via the restriction sites Nde I and Xho I into the vector pET28a (Novagen, Darmstadt, Germany) and expressed in the E. coli expression strain BL21 (DE3) (Novagen).

50 to 100 ml of LB-medium with antibiotics were inoculated with a single E. coli colony. The cultures were grown under shaking at 37° C. until OD600 0,6-1. The induction was carried out with a final IPTG concentration of 0,4 mM overnight at 16° C. Subsequently the expression cultures were harvested via centrifugation at 4000 g for 10 min. The pellet was resuspended in lysis buffer (50 mM NaH₂PO₄ pH 8,0; 300 mM NaCl; 10 mM imidazol) and incubated at 4° C. for 30 min after the addition of 1 mg/ml lysozyme. Cell disruption was carried with sonication in 6 cycles at each time for 10 sec with 80 W. RNase A (10 μg/ml) and DNase I (5 μg/ml) were added and the probes were incubated for another 10 min at 4° C. The supernatant of centrifugation at 10000 g for 30 min comprised the soluble protein fraction.

4 ml of soluble protein fraction was supplied with 1 ml of 50% Ni-NTA agarose (Qiagen, Hilden, Germany) and incubated under shaking for 1 h at 4° C. Subsequently the whole sample was added to a Polypropylene column (Qiagen) and the flow-through was saved. Up to three washing steps with every 4 ml washing buffer (50 mM NaH₂PO₄ pH 8,0; 300 mM NaCl; 20 mM imidazol) were carried out. The elution of proteins was carried out in four steps with every 0,5 ml of elution buffer (50 mM NaH₂PO₄ pH 8,0; 300 mM NaCl; 250 mM imidazol).

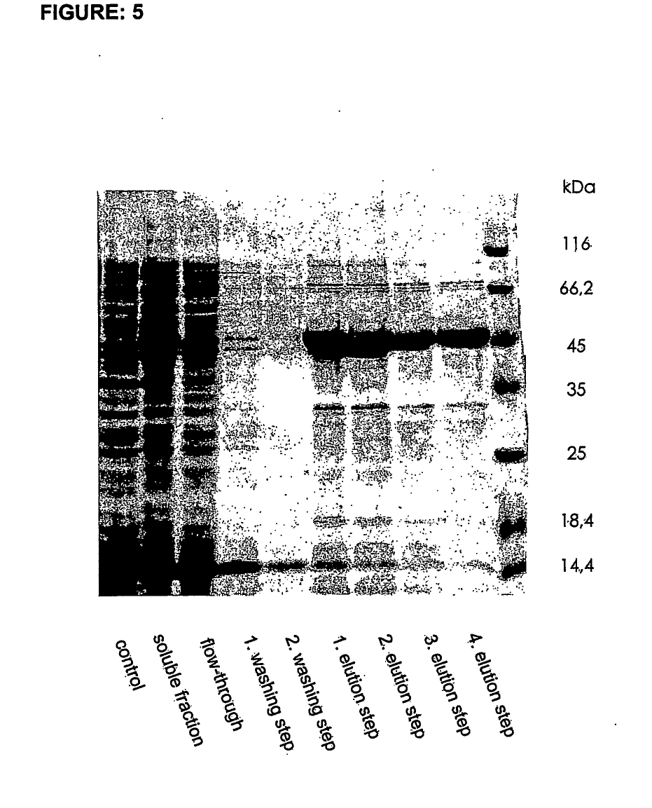

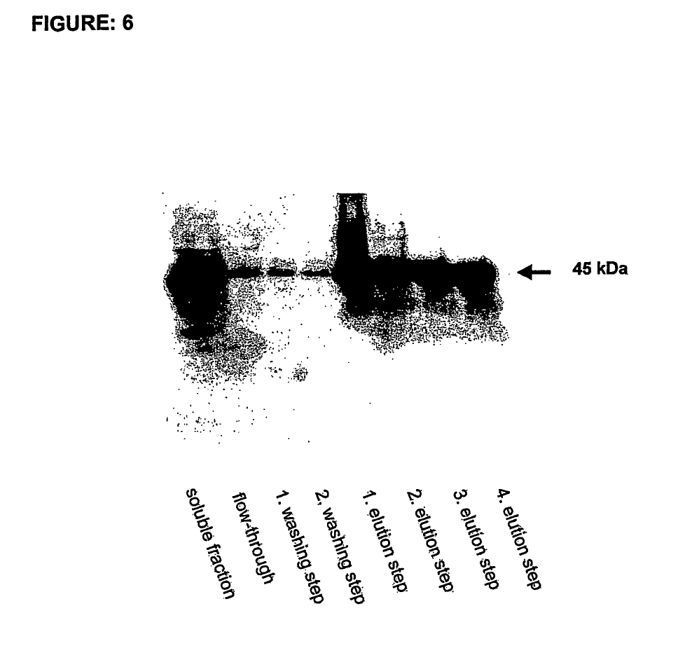

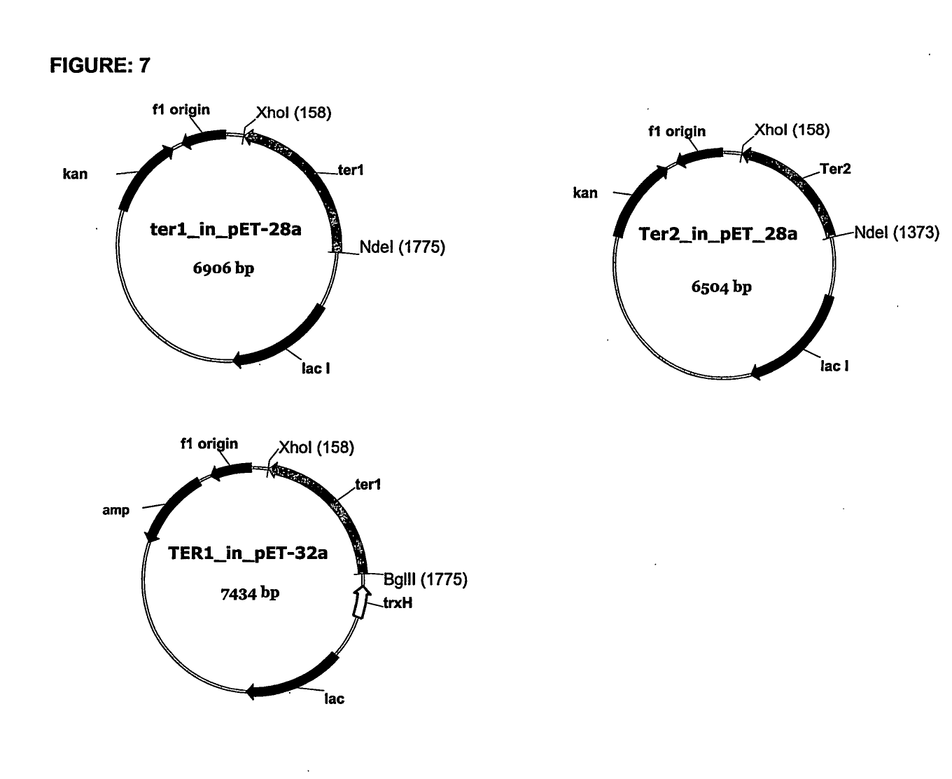

SDS-PAGE of soluble fraction of E.coli cells transformed with ter2-pET28 shows a major band at the expected size of 45 kDA in contrast to control cells (see FIG. 5). The ter2-protein was purified with the help of the added His-Tag via Ni-NTA agarose and 12% SDS-PAGE showed strong enrichment of ter2-protein (see FIG. 5). Western blot analysis and immunodetection were carried out using standard protocols (Sambrook et al., 1989, Cold Spring Harbor Laboratory Press: ISBN 0-87969-3094). As first antibody a monoclonal mouse IgG His-Tag antibody (Novagen, Darmstadt, Germany) was used and the second antibody was a HRP-conjugated anti-mouse antibody from goat (Amersham Biosciences, Freiburg, Germany). Detection of secondary antibody was carried out with ECL Western Blotting Analysis Kit (Amersham Biosciences). The Western blot analysis of ter2-pET28 construct in BL21 (DE3) with subsequent immunodetection of the His-Tag revealed specific signals of the overexpressed ter2-protein (see FIG. 6 and 7). Specific activity of purified ter2-protein was 1510 nmol·mg−1·min−1.

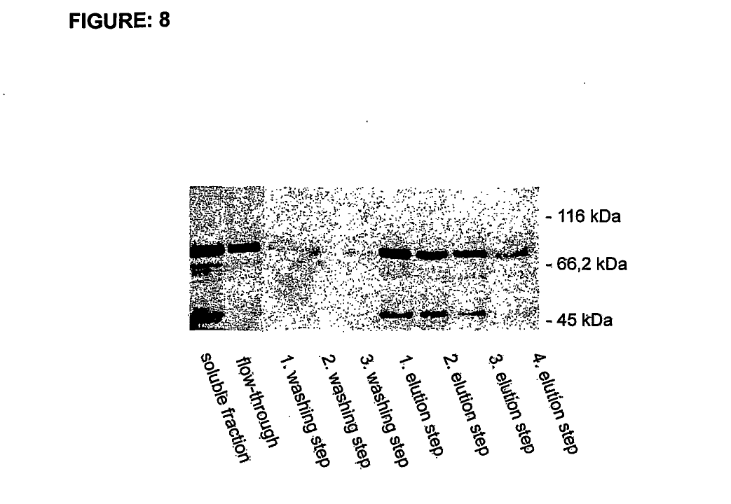

ter1 was inserted in the vector pET28a (Novagen) and pET32a (Novagen) via the resriction sites Nde I and Xho I and respectively Bgl II and Xho I (see FIGS. 7 and 8). No expression could be shown for ter1 in E. coli BL21 (DE3) see FIG. 8 western blot of ter1-pET32 construct in Rosetta(DE3). Therefore the E. coli expression host strains Origami(DE3) and Rosetta(DE3) (both Novagen) were tested for expression of ter1. In the SDS-PAGE for the ter1-constructs in pET28a no expression could be shown. However, Western blotting with subsequent immunodectection of the His-Tag showed a specific signal for the construct ter1-pET32 in E. coli Rosetta(DE3) (see FIG. 8). The signal showed the expected size of 78 kDa of the ter1-pET32 construct including the thioredoxin-Tag of 19 kDa. A TER-specific activity for the expressed ter1-protein could not be measured.

The results of the expression studies of Euglena gracilis TER in E. coli can be taken as consideration that the N-terminal part of the cDNA clone may constitute a mitochondrial targeting signal, which has to be cleaved to yield the mature and active TER protein. Nevertheless the possibility that the N-terminal part of the cDNA clone constitutes a transmembrane domain can not be excluded. This possible transmembrane domain could be lost during biochemical purification of TER from Euglena gracilis (see example 1). If expressed in E. coli this domain may possibly disrupt activity measurement with the C4-substrat (see example 1) due to incorrect convolution or missing membrane-linkage.

### Example 7

Construction of a set of TER constructs for overexpression in Arabidopsis

Since the shortened version ter2 of the Ter gene was active in E.coli, shortened and modified, but functional versions of the TER may be produced. As an example two shortened versions of the Ter gene could be generated by PCR, sequenced and also transformed into plants: One with an ORF given by SEQ ID NO: 4 resulting in the protein sequence given by SEQ ID NO: 5. This protein sequence is shortened compared to SEQ ID NO: 2 but identical to amino acid residues 126 and the following of SEQ ID NO: 2. However before this identical region it has a 28 amino acid N-terminal stretch differing to SEQ ID NO: 2. This 28 amino acids is predicted to result in mitochondrial targeting without the need for the 125 amino acid long putative mitochondrial targeting sequence of SEQ ID NO: 2.

The second sequence (SEQ ID NO: 6) results in the amino acid sequence SEQ ID NO: 7 and corresponds to the shortened version described above as ter2 and shown to be active in E.coli. The protein resulting from this sequence is not predicted to be targeted to the mitochondrium.

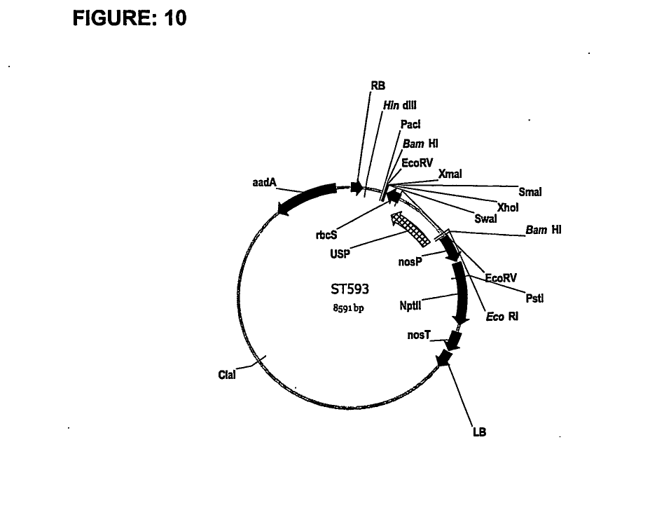

As another example of variations ter 1 (SEQ ID NO: 1) and ter2 (SEQ ID NO: 6) may be cloned into the binary vector ST593 (see FIG. 10) so that a new N-terminal sequence is added to the ORF, resulting in SEQ ID NO: 8 and 10 respectively. The corresponding protein sequences (SEQ ID NO: 9 and 11) will be the plastid targeting peptide of the small subunit of Rubisco fused to ter1 and ter 2 respectively.

These DNA sequences could be cloned into the binary vectors as exemplified below in example 8.

### Example 8

Plasmids for Plant Transformation

For plant transformation binary vectors such as pBinAR can be used (Höfgen & Willmitzer 1990, Plant Sci. 66:221-230). Construction of the binary vectors can be performed by ligation of the cDNA in sense or antisense orientation into the T-DNA. 5-prime to the cDNA a plant promoter activates transcription of the cDNA. A polyadenylation sequence is located 3′-prime to the cDNA. Tissue-specific expression can be achieved by using a tissue specific promoter. For example, seed-specific expression can be achieved by cloning the napin or LeB4 or USP promoter 5-prime to the cDNA. Also any other seed specific promoter element can be used. For constitutive expression within the whole plant the CaMV 35S promoter can be used. The expressed protein can be targeted to a cellular compartment using a signal peptide, for example for plastids, mitochondria or endoplasmic reticulum (Kermode 1996, Crit. Rev. Plant Sci. 15:285-423). The signal peptide is cloned 5-prime in frame to the cDNA to achieve subcellular localization of the fusion protein.

Further examples for plant binary vectors are the pSUN300 vectors into which the TER gene candidates is cloned, see FIG. 4. These binary vectors contain an antibiotic resistance gene driven under the control of the Nos-promotor, and a USP seed-specific promoter in front of the candidate gene with the OCS terminator. TER cDNA is cloned into the multiple cloning site of the plant binary vector in sense or antisense orientation behind the USP seed-specific promoter. The recombinant vector containing the gene of interest is transformed into Dh5α (Invitrogen) using standard conditions. Transformed cells are selected for on LB agar containing 100 μg/ml streptomycin grown overnight at 37° C. Plasmid DNA is extracted using the QIAprep Spin Miniprep Kit (Qiagen) following manufacturer's instructions. Analysis of subsequent clones and restriction mapping is performed according to standard molecular biology techniques (Sambrook et al. 1989, Molecular Cloning, A Laboratory Manual. 2^{nd} Edition. Cold Spring Harbor Laboratory Press. Cold Spring Harbor, N.Y.).

### Example 9

Transformation of Arabidopsis

One skilled in the art may choose from various methods, the following one is given as an example:

Using floral dip essentially as described by Clough and Bent, 1998, plants are transformed with Agrobacterium tumefaciens C58C1 harboring the plasmid pSUN300-USP-TER-OCS respectively. Entire plants (inflorescence and rosette) are submerged for 20-30 sec in the infiltration media consisting of 5% sucrose, 0.02% Silwet L-77 (Osi Specialties, Danbury, Conn.) and re-suspended transformed A. tumefasciens cells. Plants are then transferred to a growth chamber with a photoperiod of 16 h of light at 21° C. and 8 h of dark at 18° C. (70% humidity) or a similarly air-conditioned greenhouse. The T1 seeds are collected from mature plants. Subsequently transformed plants were identified on selection media by growing T1 seeds on MS-agar plates supplemented with kanamycin (50 μg/ml).

### Example 10

In vitro Analysis of the Function of the TER gene in Transgenic Plants

The determination of activities and kinetic parameters of enzymes is well established in the art. TER activity can be measured according to Inui and co-workers (Inui et al., 1984).

### Example 11

Lipid content in transgenic Arabidopsis plants over-expressing the TER gene.

Plant lipids were extracted from plant material as described by Cahoon et al. (1999, Proc. Natl. Acad. Sci. USA 96, 22:12935-12940) and Browse et al. (1986, Anal, Bio-chemistry 442:141-145). Qualitative and quantitative lipid or fatty acid analysis is described in Christie, William W., Advances in Lipid Methodology. Ayr/Scotland :Oily Press.—(Oily Press Lipid Library; Christie, William W., Gas Chromatography and Lipids. A Practical Guide—Ayr, Scotland :Oily Press, 1989 Repr. 1992.—IX,307).

The total lipid content of dry T2 generation seeds from Arabidopsis thaliana plants over-expressing SEQ ID NO: 1, SEQ ID NO: 4 or SEQ ID NO: 6 will be increased compared to the empty vector Arabidopsis thaliana control plants not expressing SEQ ID NO: 1, SEQ ID NO: 4 or SEQ ID NO: 6. As an example the T2 seed lipid content determined by conventional gas chromatography as mg fatty acids per mg dry seeds of the plants overexpressing SEQ ID NO: 4 is given in table 4. Each value is the mean of two separate extractions of the seed of the given line. The control value is the mean of the seeds of 4 empty vector control plants grown simultaneously, each extracted and measured also in duplicate.

TABLE 4
Total seed lipids after overexpression of SEQ ID NO: 4

| Line | % fatty acids |
|---|---|
| 22 | 31.4 ± 0.9 |
| 10 | 32.4 ± 1.1 |
| 7 | 35.5 ± 3.2 |
| controls | 28.3 ± 0.4 |

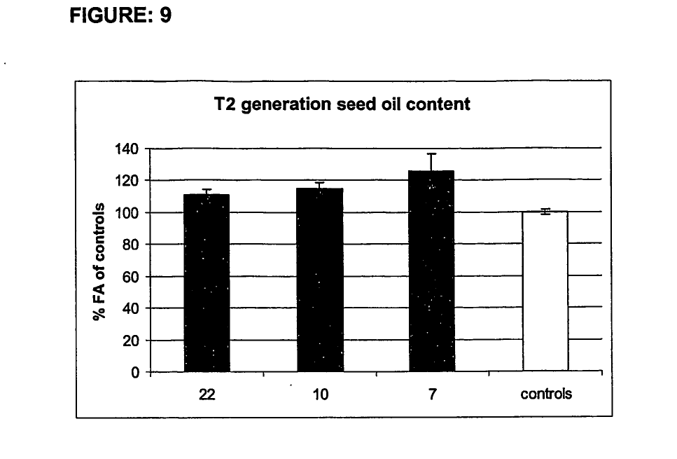

When expressed as a relative seed lipid content compared to the control plants carrying the empty pSun300-USP vector, a significant increase was seen as shown in FIG. 9. FIG. 9 describes seed oil content of Arabidopsis thaliana T2 (grey bars) of plants expressing SEQ ID NO: 4 under the control of a seed specific promoter. The white bar represents the T2 controls. Error bars represent the standard deviation of 2 independent extractions for T2 seeds. All values are shown as percentage of the average of the corresponding control plants. The independent lines showed an increase in seed storage lipids of at least 10% compared to the controls.

### Example 12

Amino acid residues characteristic for trans-enoyl-activity based on sequence comparison

Table 3 shows an alignment of the TER protein sequence with the closest sequences found. On the other hand sequence comparison with plastid enoyl-ACP-reductase sequences from public databases shows little overall homology, but hints to some amino acid residues that may be conserved. Table 4 below lists the amino acid residues of the TER (SEQ ID NO: 2) that were found to be conserved in plant plastidial enoyl-ACP-reductases as well as in Euglena trans-enoyl-CoA-reductase and the sequence homologues shown in table 3. The amino acid residues shown in table 5 appear connected to the enoyl reductase function independent of ACP or CoA dependency. The relative positions are shown, but one skilled in the art has to expect that variations of 5 to 10 amino acids, in some cases 20 to 30 amino acids in the relative positions of these key residues will occur in some members of the enzyme family.

TABLE 5
Amino acid residues connected to enoyl reductase function

| | Position in TER | |
|---|---|---|
| Amino acid residue | SEQ ID NO: 1 | relative positions |
| G | 194 | 0 |
| (R/K) | 248 | 54 |
| (L/I) | 250 | 56 |
| K/R | 260 | 66 |
| V/I | 275 | 81 |
| (T/S) | 291 | 97 |
| (V/I/L) | 311 | 117 |
| Y | 367 | 173 |
| Y | 377 | 183 |
| (G) | 380 | 186 |
| V | 408 | 214 |
| (S) | 417 | 223 |

Amino acids separated by a slash are interchangeably found at this position. Amino acids listed in brackets are found in the large majority of sequences.

1. An isolated polypeptide having trans-2-enoyl-CoA reductase (TER) (EC 1.3.1.44) activity comprising a polypeptide sequence selected from the group comprising
a) a sequence as described by SEQ ID NO: 2, SEQ ID NO: 5, SEQ ID NO: 7, SEQ ID NO: 9 or SEQ ID NO: 11,
b) a sequence having an identity of at least 60% to a sequence as described by SEQ ID NO: 2, SEQ ID NO: 5, SEQ ID NO: 7, SEQ ID NO: 9 or SEQ ID NO: 11, and
c) a sequence comprising at least 10 consecutive amino acid residues of a sequence as described by SEQ ID NO: 2.

2. An isolated nucleic acid molecule comprising a sequence selected from the group consisting of
a) a sequence as described by SEQ ID NO: 1, SEQ ID NO: 4, SEQ ID NO: 6, SEQ ID NO: 8or SEQ ID NO: 10,
b) a sequence encoding a polypeptide as claimed in claim 1, and
c) sequences which under stringent conditions hybridise with a sequence encoding a polypeptide as claimed in claim 1.

3. A method of increasing the total oil content in a plant organism or a tissue, organ, part, cell or propagation material thereof, comprising
a) expressing a polynucleotide SEQ ID NO: 1, SEQ ID NO: 4, SEQ ID NO: 6, SEQ ID NO: 8 or SEQ ID NO: 10 in said plant organism or in a tissue, organ, part, cell or propagation material thereof, and
b) selecting a plant organism in which—in contrast to or comparison with a starting organism—the total oil content in said plant organism or in a tissue, organ, part, cell or propagation material thereof is increased.

4. The method as claimed in claim 3, wherein the TER protein is encoded by a nucleic acid sequence selected from the group consisting of:
a) a nucleic acid sequence comprising a nucleotide sequence which is at least 60% identical to the nucleic acid sequence of SEQ ID NO: 1, SEQ ID NO: 4, SEQ ID NO: 6, SEQ ID NO: 8or SEQ ID NO: 10;
b) a nucleic acid sequence comprising a fragment of at least 30 nucleotides of a nucleic acid sequence comprising the nucleotide sequence of SEQ ID NO: 1, SEQ ID NO: 4, SEQ ID NO: 6, SEQ ID NO: 8 or SEQ ID NO: 10;
c) a nucleic acid sequence which encodes a polypeptide comprising an amino acid sequence at least about 60% identical to the amino acid sequence of SEQ ID NO: 2, SEQ ID NO: 5, SEQ ID NO: 7, SEQ ID NO: 9 or SEQ ID NO: 11 and
d) a nucleic acid sequence which encodes a fragment of a polypeptide comprising the amino acid sequence of SEQ ID NO: 2, SEQ ID NO: 5, SEQ ID NO: 7, SEQ ID NO: 9 or SEQ ID NO: 11 or wherein the fragment comprises at least 10 contiguous amino acid residues of the amino acid sequence of SEQ ID NO: 2.

5. The method as claimed in claim 3, wherein the plant is an oil crop.

6. The method as claimed in claim 5, wherein the total oil content in seed of a plant is increased.

7. An expression cassette comprising in combination with a regulatory sequence a nucleic acid sequence selected from the group consisting of:
a) a nucleic acid sequence comprising a nucleotide sequence which is at least 60% identical to the nucleotide sequence of SEQ ID NO: 1, SEQ ID NO: 4, SEQ ID NO: 6, SEQ ID NO: 8 or SEQ ID NO: 10,
b) a nucleic acid sequence comprising a fragment of at least 30 nucleotides of a nucleic acid sequence comprising the nucleotide sequence of SEQ ID NO: 1, SEQ ID NO: 4, SEQ ID NO: 6,SEQ ID NO: 8 or SEQ ID NO: 10,
c) a nucleic acid sequence which encodes a polypeptide comprising an amino acid sequence at least about 60% identical to the amino acid sequence of SEQ ID NO: 2, SEQ ID NO: 5, SEQ ID NO: 7, SEQ ID NO: 9 or SEQ ID NO: 11, or
d) a nucleic acid sequence which encodes a fragment of a polypeptide comprising the amino acid sequence of SEQ ID NO: 2, SEQ ID NO: 5, SEQ ID NO: 7, SEQ ID NO: 9 or SEQ ID NO: 11 wherein the fragment comprises at least 10 contiguous amino acid residues of the amino acid sequence of SEQ ID NO: 2,
wherein said regulatory sequence is capable of mediating expression of said nucleic acid sequence in a plant.

8. An expression cassette according to claim 7, wherein said nucleic acid sequence encodes a polypeptide comprising the amino acid sequence set forth in SEQ ID NO: 2, SEQ ID NO: 5, SEQ ID NO: 7, SEQ ID NO: 9 or SEQ ID NO: 11.

9. An expression cassette as claimed in claim 7, wherein the promoter is a seed-specific promoter.

10. A genetically modified plant organism or tissue, organ, part, cell or propagation material thereof, comprising a polypeptide as defined in SEQ ID NO: 2, SEQ ID NO: 5, SEQ ID NO: 7, SEQ ID NO: 9 or SEQ ID NO: 11 or an expression cassette as claimed in claim 7.

11. The genetically modified plant organism as claimed in claim 10, wherein the plant organism is selected from the group of the oil crops consisting of Borvago officinalis, Brassica campestris, Brassica napus, Brassica rapa, Cannabis sativa, Carthamus tinctorius, Cocos nucifera, Crambe abyssinica, Cuphea species, Elaeis guinensis, Elaeis oleifera, Glycine max, Gossypium hirsutum, Gossypium barbadense, Gossypium herbaceum, Helianthus annuus, Linum usitatissimum, Oenothera biennis, Olea europaea, Oryza sativa, Ricinus communis, Sesamum indicum, Triticum species, Zea mays, walnut and almond.

12. A method for the production of triacylglycerols, diacylglycerols, monoacylglycerols, phospholipids, waxesters and/or fatty acids or derivatives of the above comprising using a genetically modified plant organism or tissue, organ, part, cell or propagation material thereof as claimed in claim 10.


SEQUENCE LISTING (55 sequences)

SEQ ID NO: 1 - DNA, 1620 bp, Euglena gracilis
  CDS (1)..(1620)
atgtcgtgccccgcctcgccgtctgctgccgtggtgtctgccggcgccctctgcctgtgcgtggcaacggtattgttggcgactggatccaaccccaccgccctgtccactgcttccactcgctctccgacctcactggtccgtggggtggacaggggcttgatgaggccaaccactgcagcggctctgacgacaatgagagaggtgccccagatggctgagggattttcaggcgaagccacgtctgcatgggccgccgcggggccgcagtgggcggcgccgctcgtggccgcggcctcctccgcactggcgctgtggtggtgggccgcccggcgcagcgtgcggcggccgctggcagcgctggcggagctgcccaccgcggtcacccacctggcccccccgatggcgatgttcaccaccacagcgaaggtcatccagcccaagattcgtggcttcatctgcacgaccacccacccgatcggctgtgagaagcgggtccaggaggagatcgcgtacgcccgtgcccacccgcccaccagccctggcccgaagagggtgctggtcatcggctgcagtaccggctacgggctctccacccgcatcaccgctgccttcggctaccaggccgccacgctgggcgtgttcctggcgggccccccgacgaagggccgccccgccgcggcgggctggtacaacaccgtggcgttcgagaaggccgccctggaggccgggctgtacgcccggagccttaatggcgacgccttcgactccacaacgaaggcgcggacggtcgaggcgatcaagcgggacctcggcacggtggacctcgtggtgtacagcatcgccgccccgaagcggacggaccctgccaccggcgtcctccacaaggcctgcctgaagcccatcggcgccacgtacaccaaccgcactgtgaacaccgacaaggcggaggtgaccgacgtcagcattgagccggcctcccccgaagagatcgcggacacggtgaaggtgatgggcggggaggactgggagctctggatccaggcgctgtcggaggccggcgtgctggcggagggggccaagacggtggcgtactcctacatcggccccgagatgacgtggcctgtctactggtccggcaccatcggggaggccaagaaggacgtggagaaggctgccaagcgcatcacgcagcagtacggctgcccggcgtacccggtggtggccaaggccttggtcacccaggccagctccgccatcccggtggtgccgctctacatctgcctgctgtaccgcgttatgaaggagaagggcacccacgagggctgcatcgagcagatggtgcggctgctcaccacgaagctgtaccccgagaacggggcccccatcgtcgatgaggccggacgtgtgcgggtggatgactgggagatggcggaggatgtgcagcaggctgttaaggacctctggagccaggtgagcactgccaacctcaaggacatctccgacttcgctgggtatcaaactgagttcctgcggctgttcgggttcggcattgacggcgtggactacgaccagcccgtggacgtggaggcggacctccccagtgctgcccagcagtag

SEQ ID NO: 2 - protein, 539 aa, Euglena gracilis
MSCPASPSAAVVSAGALCLCVATVLLATGSNPTALSTASTRSPTSLVRGVDRGLMRPTTAAALTTMREVPQMAEGFSGEATSAWAAAGPQWAAPLVAAASSALALWWWAARRSVRRPLAALAELPTAVTHLAPPMAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 3 - DNA, 837 bp, Euglena gracilis
gggtggtataatacggtggcgttcgagaaggccgccctggaggccgggctgtacgcccggagccttaatggcgacgcattcgactctacaacgaaggcgcggacggttgaggcgatcaagcgggatctcggcacggtggacctcgtggtgtacagcatcgccgccccgaaacggacggaccctgccaccggcgtcgtccacaaggcctgcctgaagcccatcggcgccacctacaccaaccgcactgtgaacaccgacaaggcggaggtgaccgatgtcagcatcgagccggcctcccccgaggagatcgccgacacggtgaaggtgatgggcggggaggactgggagctttggatccaggcactgtcggaggccggcgtgctggcggagggggccaagacggtggcatactcctacatcggccccgagatgacgtggcccgtgtactggtccggcaccattggggaggccaagaaggacgtggagaaggccgctaagcgcatcacacagcagtacggctgcccagcatacccggtggtggccaaggccttggtcacccaggccagctctgccatcccggtggtgccgctctacatctgcctgctgtaccgcgttatgaaggagaagggcacccacgagggctgcatcgagcagatggtgcggctgctcaccacgaagctgtaccccgggaacggttcccccattgtcgatgaggccgggcgggtgcgggtggatgactgggagatggcggaggatgtgcagcaggctgtgaaggacctctggagccaggtgaacactgccaacctcaaggacatttccgattttgccggctatgaa

SEQ ID NO: 4 - DNA, 1329 bp, Euglena gracilis
  CDS (1)..(1329)
atgggccgccgcggggccgcagtgggcggcgccgctcgtggccgcggcctcctccgcactggcgctgtggtggtgaacatcgccaccgcggtcacccacctggcccccccgatggcgatgttcaccaccacagcgaaggtcatccagcccaagattcgtggcttcatctgcacgaccacccacccgatcggctgtgagaagcgggtccaggaggagatcgcgtacgcccgtgcccacccgcccaccagccctggcccgaagagggtgctggtcatcggctgcagtaccggctacgggctctccacccgcatcaccgctgccttcggctaccaggccgccacgctgggcgtgttcctggcgggccccccgacgaagggccgccccgccgcggcgggctggtacaacaccgtggcgttcgagaaggccgccctggaggccgggctgtacgcccggagccttaatggcgacgccttcgactccacaacgaaggcgcggacggtcgaggcgatcaagcgggacctcggcacggtggacctcgtggtgtacagcatcgccgccccgaagcggacggaccctgccaccggcgtcctccacaaggcctgcctgaagcccatcggcgccacgtacaccaaccgcactgtgaacaccgacaaggcggaggtgaccgacgtcagcattgagccggcctcccccgaagagatcgcggacacggtgaaggtgatgggcggggaggactgggagctctggatccaggcgctgtcggaggccggcgtgctggcggagggggccaagacggtggcgtactcctacatcggccccgagatgacgtggcctgtctactggtccggcaccatcggggaggccaagaaggacgtggagaaggctgccaagcgcatcacgcagcagtacggctgcccggcgtacccggtggtggccaaggccttggtcacccaggccagctccgccatcccggtggtgccgctctacatctgcctgctgtaccgcgttatgaaggagaagggcacccacgagggctgcatcgagcagatggtgcggctgctcaccacgaagctgtaccccgagaacggggcccccatcgtcgatgaggccggacgtgtgcgggtggatgactgggagatggcggaggatgtgcagcaggctgttaaggacctctggagccaggtgagcactgccaacctcaaggacatctccgacttcgctgggtatcaaactgagttcctgcggctgttcgggttcggcattgacggcgtggactacgaccagcccgtggacgtggaggcggacctccccagtgctgcccagcagtag

SEQ ID NO: 5 - protein, 442 aa, Euglena gracilis
MGRRGAAVGGAARGRGLLRTGAVVVNIATAVTHLAPPMAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 6 - DNA, 1212 bp, Euglena gracilis
  CDS (1)..(1212)
atgttcaccaccacagcgaaggtcatccagcccaagattcgtggcttcatctgcacgaccacccacccgatcggctgtgagaagcgggtccaggaggagatcgcgtacgcccgtgcccacccgcccaccagccctggcccgaagagggtgctggtcatcggctgcagtaccggctacgggctctccacccgcatcaccgctgccttcggctaccaggccgccacgctgggcgtgttcctggcgggccccccgacgaagggccgccccgccgcggcgggctggtacaacaccgtggcgttcgagaaggccgccctggaggccgggctgtacgcccggagccttaatggcgacgccttcgactccacaacgaaggcgcggacggtcgaggcgatcaagcgggacctcggcacggtggacctcgtggtgtacagcatcgccgccccgaagcggacggaccctgccaccggcgtcctccacaaggcctgcctgaagcccatcggcgccacgtacaccaaccgcactgtgaacaccgacaaggcggaggtgaccgacgtcagcattgagccggcctcccccgaagagatcgcggacacggtgaaggtgatgggcggggaggactgggagctctggatccaggcgctgtcggaggccggcgtgctggcggagggggccaagacggtggcgtactcctacatcggccccgagatgacgtggcctgtctactggtccggcaccatcggggaggccaagaaggacgtggagaaggctgccaagcgcatcacgcagcagtacggctgcccggcgtacccggtggtggccaaggccttggtcacccaggccagctccgccatcccggtggtgccgctctacatctgcctgctgtaccgcgttatgaaggagaagggcacccacgagggctgcatcgagcagatggtgcggctgctcaccacgaagctgtaccccgagaacggggcccccatcgtcgatgaggccggacgtgtgcgggtggatgactgggagatggcggaggatgtgcagcaggctgttaaggacctctggagccaggtgagcactgccaacctcaaggacatctccgacttcgctgggtatcaaactgagttcctgcggctgttcgggttcggcattgacggcgtggactacgaccagcccgtggacgtggaggcggacctccccagtgctgcccagcagtag

SEQ ID NO: 7 - protein, 403 aa, Euglena gracilis
MFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 8 - DNA, 1803 bp, Euglena gracilis
  CDS (1)..(1803)
atggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtatttaaatctcgagatgtcgtgccccgcctcgccgtctgctgccgtggtgtctgccggcgccctctgcctgtgcgtggcaacggtattgttggcgactggatccaaccccaccgccctgtccactgcttccactcgctctccgacctcactggtccgtggggtggacaggggcttgatgaggccaaccactgcagcggctctgacgacaatgagagaggtgccccagatggctgagggattttcaggcgaagccacgtctgcatgggccgccgcggggccgcagtgggcggcgccgctcgtggccgcggcctcctccgcactggcgctgtggtggtgggccgcccggcgcagcgtgcggcggccgctggcagcgctggcggagctgcccaccgcggtcacccacctggcccccccgatggcgatgttcaccaccacagcgaaggtcatccagcccaagattcgtggcttcatctgcacgaccacccacccgatcggctgtgagaagcgggtccaggaggagatcgcgtacgcccgtgcccacccgcccaccagccctggcccgaagagggtgctggtcatcggctgcagtaccggctacgggctctccacccgcatcaccgctgccttcggctaccaggccgccacgctgggcgtgttcctggcgggccccccgacgaagggccgccccgccgcggcgggctggtacaacaccgtggcgttcgagaaggccgccctggaggccgggctgtacgcccggagccttaatggcgacgccttcgactccacaacgaaggcgcggacggtcgaggcgatcaagcgggacctcggcacggtggacctcgtggtgtacagcatcgccgccccgaagcggacggaccctgccaccggcgtcctccacaaggcctgcctgaagcccatcggcgccacgtacaccaaccgcactgtgaacaccgacaaggcggaggtgaccgacgtcagcattgagccggcctcccccgaagagatcgcggacacggtgaaggtgatgggcggggaggactgggagctctggatccaggcgctgtcggaggccggcgtgctggcggagggggccaagacggtggcgtactcctacatcggccccgagatgacgtggcctgtctactggtccggcaccatcggggaggccaagaaggacgtggagaaggctgccaagcgcatcacgcagcagtacggctgcccggcgtacccggtggtggccaaggccttggtcacccaggccagctccgccatcccggtggtgccgctctacatctgcctgctgtaccgcgttatgaaggagaagggcacccacgagggctgcatcgagcagatggtgcggctgctcaccacgaagctgtaccccgagaacggggcccccatcgtcgatgaggccggacgtgtgcgggtggatgactgggagatggcggaggatgtgcagcaggctgttaaggacctctggagccaggtgagcactgccaacctcaaggacatctccgacttcgctgggtatcaaactgagttcctgcggctgttcgggttcggcattgacggcgtggactacgaccagcccgtggacgtggaggcggacctccccagtgctgcccagcagtag

SEQ ID NO: 9 - protein, 600 aa, Euglena gracilis
MASMISSSAVTTVSRASTVQSAAVAPFGGLKSMTGFPVKKVNTDITSITSNGGRVKYLNLEMSCPASPSAAVVSAGALCLCVATVLLATGSNPTALSTASTRSPTSLVRGVDRGLMRPTTAAALTTMREVPQMAEGFSGEATSAWAAAGPQWAAPLVAAASSALALWWWAARRSVRRPLAALAELPTAVTHLAPPMAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 10 - DNA, 1389 bp, Euglena gracilis
  CDS (1)..(1389)
atggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtatttaaatatgttcaccaccacagcgaaggtcatccagcccaagattcgtggcttcatctgcacgaccacccacccgatcggctgtgagaagcgggtccaggaggagatcgcgtacgcccgtgcccacccgcccaccagccctggcccgaagagggtgctggtcatcggctgcagtaccggctacgggctctccacccgcatcaccgctgccttcggctaccaggccgccacgctgggcgtgttcctggcgggccccccgacgaagggccgccccgccgcggcgggctggtacaacaccgtggcgttcgagaaggccgccctggaggccgggctgtacgcccggagccttaatggcgacgccttcgactccacaacgaaggcgcggacggtcgaggcgatcaagcgggacctcggcacggtggacctcgtggtgtacagcatcgccgccccgaagcggacggaccctgccaccggcgtcctccacaaggcctgcctgaagcccatcggcgccacgtacaccaaccgcactgtgaacaccgacaaggcggaggtgaccgacgtcagcattgagccggcctcccccgaagagatcgcggacacggtgaaggtgatgggcggggaggactgggagctctggatccaggcgctgtcggaggccggcgtgctggcggagggggccaagacggtggcgtactcctacatcggccccgagatgacgtggcctgtctactggtccggcaccatcggggaggccaagaaggacgtggagaaggctgccaagcgcatcacgcagcagtacggctgcccggcgtacccggtggtggccaaggccttggtcacccaggccagctccgccatcccggtggtgccgctctacatctgcctgctgtaccgcgttatgaaggagaagggcacccacgagggctgcatcgagcagatggtgcggctgctcaccacgaagctgtaccccgagaacggggcccccatcgtcgatgaggccggacgtgtgcgggtggatgactgggagatggcggaggatgtgcagcaggctgttaaggacctctggagccaggtgagcactgccaacctcaaggacatctccgacttcgctgggtatcaaactgagttcctgcggctgttcgggttcggcattgacggcgtggactacgaccagcccgtggacgtggaggcggacctccccagtgctgcccagcagtag

SEQ ID NO: 11 - protein, 462 aa, Euglena gracilis
MASMISSSAVTTVSRASTVQSAAVAPFGGLKSMTGFPVKKVNTDITSITSNGGRVKYLNMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 12 - DNA, 20 bp, Artificial sequence
  Artificial Sequence  primer
  misc_feature (3)..(3): n is inosine
  misc_feature (15)..(15): n is inosine
  misc_feature (18)..(18): n is inosine
ggntggtayaayacngtngc

SEQ ID NO: 13 - DNA, 20 bp, Artificial sequence
  Artificial sequence primer
  misc_feature (9)..(9): n is inosine
  misc_feature (12)..(12): n is inosine
gtytcrtanccngcraartc

SEQ ID NO: 14 - protein, 13 aa, Euglena gracilis
ACLKPLGATYTNR

SEQ ID NO: 15 - protein, 10 aa, Euglena gracilis
AALEAGLYAR

SEQ ID NO: 16 - protein, 15 aa, Euglena gracilis
VLVLGCSTGYGLSTR

SEQ ID NO: 17 - protein, 5 aa, Euglena gracilis
TDPAT

SEQ ID NO: 18 - protein, 12 aa, Euglena gracilis
SLDGDAFDSTTK

SEQ ID NO: 19 - protein, 12 aa, Euglena gracilis
DLWSQVNTANLK

SEQ ID NO: 20 - protein, 11 aa, Euglena gracilis
AGWYNTVAFEK

SEQ ID NO: 21 - protein, 10 aa, Euglena gracilis
RVQEELAYAR

SEQ ID NO: 22 - protein, 14 aa, Euglena gracilis
DLSDFAGYQTEFLR

SEQ ID NO: 23 - protein, 15 aa, Euglena gracilis
LYPGDGSPLVDEAGR

SEQ ID NO: 24 - protein, 15 aa, Euglena gracilis
LTQQYGCPAYPVVAK

SEQ ID NO: 25 - protein, 15 aa, Euglena gracilis
VDDWEMAEDVQQAVK

SEQ ID NO: 26 - protein, 9 aa, Euglena gracilis
  misc_feature (6)..(9): Xaa from positions 6 to 9 are Ala Met Val Arg or Leu Ser Glu Lys
STGYGXXXX

SEQ ID NO: 27 - protein, 10 aa, Euglena gracilis
AHPPTSPGPK

SEQ ID NO: 28 - protein, 12 aa, Euglena gracilis
ALSEAGVLAEQK

SEQ ID NO: 29 - protein, 12 aa, Euglena gracilis
  misc_feature (1)..(2): Xaa at positions 1 and 2 are Gly Thr or Ala Ser
XXHEGCLEQMVR

SEQ ID NO: 30 - protein, 14 aa, Euglena gracilis
LYPENGAPLVDEQR

SEQ ID NO: 31 - DNA, 32 bp, Artificial sequence
  Artificial sequence primer
tatacatatgtcgtgccccgcctcgccgtctg

SEQ ID NO: 32 - DNA, 35 bp, Artificial sequence
  Artificial sequence primer
tatagatcttatgtcgtgccccgcctcgccgtctg

SEQ ID NO: 33 - DNA, 35 bp, Artificial sequence
  Artificial sequence primer
tatacatatgttcaccaccacagcgaaggtcatcc

SEQ ID NO: 34 - DNA, 29 bp, Artificial sequence
  Artificial sequence primer
tatctcgagctactgctgggcagcactgg

SEQ ID NO: 35 - protein, 391 aa, Pseudomonas putida
MAIIHPKVRGFICTTTHPKGCELNVRDQIEATRKLGVREDGPKKVLVIGASSGYGLAARITAAFGFKADTLGVFFEKPGTETKAGTAGWYNAAAFDKFAKAEGLYSKSINGDAFSDEARAKVIELIKNEMGGKVDLVIYSLASPVRKLPQTGEVIRSALKPIGQPYKSTAIDTNKDTIIEASIEPATEQEIADTVTVMGGQDWQLWIDALAGANVLAEGARTVAFSYIGSDITWPIYWHGALGQAKQDLDETALRLNQKLAGEVKGGANVAVLKSVVTQASSAIPVMPLYLSMVFKIMQEKGVHEGTQDQLDRMYRDRMYRTDGAPAEVDEKGRLRLDDWELRDDVQNACKALWPQVTTENLFELTDYAGYKKQFLNLFGFERADVDYDKD

SEQ ID NO: 36 - protein, 389 aa, Vibrio vulnificus
MRIEPIIQGVVARSAHPFGCEAAIKKQIAFVKNAPQISQGPKRVLILGASSGFGLAARIALTFGGAQADTIGVSFERGPSEKGTGSAGWYNNVFFKREAEKEGRIAINIVGDAFASETRTQVIEAIETYFEGEVDLVIYSLATGMRPISNQPGEFWRSVIKPFGQTVTGASFDLEHDRWIDTTLESATEEEALHTIKVMGGEDWESWIDTLINAESIAQGCQTIAFSYVGPEITHPIYLDGTLGRAKIDLHQTSHSLNLKLANFDGAAYATVCKALVTKASVFIPALSPYLLALYRVMKDEKCHEGCIEQMQRLFATKLYGQDHISVDGERLVRMDDWELAPHIQNKVNQILEEMDANNFQVIGDYQGFKNEFLQLNGFGFDEVDYSQD

SEQ ID NO: 37 - protein, 388 aa, Vibrio vulnificus
MIIKPKIRGFICTTTHPVGCEANVKEQIAYTKAQGPIKNAPKRVLVVGASSGYGLSSRIAAAFGGGAATIGVFFEKEGSEKKPGTAGFYNAAAFEKLAREEGLYAKSLNGDAFSNEAKQKTIDLIKEDLGQVDMVVYSLASPVRKLPETGELIRSALKPIGQTYTSTAVDTNKDIIIEASVEPATEQEIQDTVTVMGGEDWELWINALAEAGVLADGCKTVAYSYIGTELTWPIYWDGALGKAKMDLDRAASALNDKLSATGGSANVAVLKSVVTQASSAIPVMPLYIAMVFKKMREEGVHEGCMEQIYRMFSQRLYKEDGSAAEVDEKNRLRLDDWELRDDIQEHCRNLWPQITTENLKELTDYVEYKEEFLKLFGFGIDGVDYEAD

SEQ ID NO: 38 - protein, 388 aa, Shewanella oneidensis
MIIKPKIRGFICTTTHPVGCEANVQEQIALTKAKGKIANGPKKVLVVGSSSGYGLSSRIAAAFGSDAATIGVFFEKPGTEAKPGTAGWYNSAAFDKFAKAEGLYSKSINCDAFSHEAKQKVIELIKQDLGQVDMVVYSLASPVRKLPDSGELVRSALKPIGETYTATAVDTNKDCIIEATVEPATEQEIADTVTVMGGQDWELWINALAEAGVLSDNCKTVAYSYIGTELTWPIYWHGALGKAKMDLDRAAKALNDKLSVTGGSANVAVLKSVVTQASSAIPVMPLYIAMVFKKMRQEGLHEGCMEQIYRMFSERLFRADGAKPETDSDNRLRLDDWELREDIQQHCRDLWPQVTTENLSELTDYQEYKAEFIKLFGFGIEGINYDAD

SEQ ID NO: 39 - protein, 389 aa, Xanthomonas campestris (pv. campestris)
MIIHPKVRGFICTTTHPLGCERNVLDQIAATRARGVRNDGPKKVLVIGASSGYGLASRITAAFGFGADTLGVFFEKPGSDKKAGTAGWYNSAAFDTHAKAAGLYSKSINGDAFSDEARAKVIELIKTDMGGQVDLVVYSLASPVRKLPSTGEVKRSALKPIGNTYTATAIDTNKDTIIQASIEPATEQEIEDTITVMGGQDWELWIDALDSAGVLAKGARSVAFSYIGTEITWPIYWHGALGKAKVDLDHTAQRLDARLQASGGGANVAVLKSVVTQASAAIPVMPLYISMVYKIMKEKGLHEGTIEQLDRLFRERLYREDGQPAEVDEQNRLRLDDWELRDDVQDACKALWPQVTTENLFALTDYAGYKHEFLKLFGFERKDVDYDAD

SEQ ID NO: 40 - protein, 389 aa, Xanthomonas axonopodis (pv. citri)
MIIHPKVRGFICTTTHPLGCERNVLEQIAATRARGVRTDGPKKVLVIGASSGYGLASRITAAFGFGADTLGVFFEKPGTASKAGTAGWYNSAAFDKHAKAAGLYSKSINGDAFSDAAREKVIELIKTEMGGQVDLMVYSLASPVRKLPGSGEVKRSALKPIGQTYTATAIDTNKDTIIQASIEPASAQEIEDTVTVMGGQDWELWIDALEGAGVLADGARSVAFSYIGTEITWPIYWHGALGKAKVDLDHTAQRLNARLARRGGGANVAVLKSVVTQASAAIPVMPLYISMVYKIMKEKGLHEGTIEQLDRLFRERLYREDGQPAQVDDENRLRLDDWELRDDVQDACKALWPQVTTENLFELTDYAGYKHEFLKLFGFERTDVDYDAD

SEQ ID NO: 41 - protein, 383 aa, Clostridium perfringens
MIVEPKFRGFICTTSHPIGCKKNVENQIEYVKENGKIEGAKRVLVLGASTGYGLASAIVASEACDAEVLGVSFEREAKGKRTASAGWYNIESLKKFVEGEGKKFISVNGDAFSNEVKSEVIDLIKENMGKVDLVIYSLAAPKRKDPVSGEVYSSCLKTVGAPFTSKTLDFHTGEIQNITINPATEEEIEGTRKVMGGEDWMLWIEALKEANVLENGVKTIAYSYIGPEVTYPIYREGTIGRAKNDLEKTAGEITKVLKSLNGEGYISVNKALVTQASSAIPIVSLYISILYKVMKEKGTHEGCIEQIYRMFKELYEGNLNLDSENRIRIDDLEMAEDVQKAIEEIWPQITSENVFELSDAEDFKKEFFKLFGFGLEGVDYSED

SEQ ID NO: 42 - protein, 429 aa, Streptomyces avermitilis
MCVWTPRRCSTGRHRTTWSSTSSRHCSTTDPAEEPMMPPSMTQSERVVKPTGRGYLLLDAHPVGCFRSVELMRAEVPVPEKPPARRPTALVIGSSSGYGLASTIAGLVRYGIDGVGIGLERPAGHRSATAGWYRTVATDAIARELGADFSFRNADAFADTTKTETLDLLAERFGGVDYLIYSVAAPRRTDPRSGTTYQSVLKPLGAPHTTRNLEFADDGAAQVREVTVAPATEAEAAATVGVMGGEDWSRWITALAERGLLRSGFRTVALTYIGSPLTSAIYRGGTIGAAKAHLESTARALTERLAAVDGRAFTSVNGALVTQALTAIPGIPLYVSLLRGVLGDRFPSPVAQSLDLWHQLTARRPDVDDSGRIRLDRWELSEPVQAAVAERWRSITPETVTALADTAWFRAQCRALYGFDVPGVDYTVP

SEQ ID NO: 43 - protein, 385 aa, Pseudomonas aeruginosa
MIIKPRVRGFICVTTHPAGCEANVKQQIDYVEAKGPVVNGPKKVLVIGSSTGYGLAARITAAFGSGADTLGVFFERPGSESKPGTAGWYNSAAFEKFAHEKGLYARSINGDAFSDEVKRLTIETIKRDLGKVDLVVYSLAAPRRTHPKSGEVFSSTLKPIGKSVSFRGLDTDKEVIKDVVLEAASDQEVADTVAVMGGEDWQMWIDALLEADVLADGAKTTAFTYLGEKITHDIYWNGSIGAAKKDLDQKVLGIRDKLAPLGGDARVSVLKAVVTQASSAIPMMPLYLSLLFKVMKEQGTHEGCIEQVDGLYRESLYGAEPRLDEEGRLRADYKELQPEVQSRVEELWDKVTNENLYELTDFAGYKSEFLNLFGFEVAGVDYEQD

SEQ ID NO: 44 - protein, 386 aa, Yersinia pestis
MIIKPRVRGFICVTAHPTGCEANVKKQIDYVTTEGPIANGPKRVLVIGASTGYGLAARITAAFGCGADTLGVFFERPGEEGKPGTSGWYNSAAFHKFAAQKGLYAKSINGDAFSDEIKQLTIDAIKQDLGQVDQVIYSLASPRRTHPKTGEVFNSALKPIGNAVNLRGLDTDKEVIKESVLQPATQSEIDSTVAVMGGEDWQMWIDALLDAGVLAEGAQTTAFTYLGEKITHDIYWNGSIGAAKKDLDQKVLAIRESLAAHGGGDARVSVLKAVVTQASSAIPMMPLYLSLLFKVMKEKGTHEGCIEQVYSLYKDSLCGDSPHMDQEGRLRADYKELDPEVQNQVQQLWDQVTNDNIYQLTDFVGYKSEFLNLFGFGIDGVDYDAD

SEQ ID NO: 45 - protein, 286 aa, Vibrio cholerae
MYAKSLNGDAFSNEAKQKAIELIKQDLGQIDLVVYSLASPVRKMPDTGELVRSALKPIGETYTSTAVDTNKDVIIEASVEPATEQEIADTVTVMGGQDWELWIQALEEAGVLAEGCKTVAYSYIGTELTWPIYWDGALGRAKMDLDRAATALNEKLAAKGGTANVAVLKSVVTQASSAIPVMPLYIAMVFKKMREQGVHEGCMEQIYRMFSQRLYKEDGSAPEVDDHNRLRLDDWELRDDIQQHCRDLWPQITTENLRELTDYDMYKEEFIKLFGFGIEGIDYDAD

SEQ ID NO: 46 - protein, 392 aa, Vibrio cholerae
MHIKPIIQGVVARSAHPYGCEQAVLQQIQYVKQANPIKSGPKRVLILGASSGFGLAARIALTFGGAQADTIGVSFERAPSETQTGSAGYYNNLFFKQHAEQAGRIAVNLEGDVFSVDMREQVIEAIETYFEGEVDLIIYSIASGMRRKPRSEKADPEFWRSAIKPIGEAVSGATLLLENDTWIETTLQPASEEEIEGTLRVMGGDDWENWIDTLINAESLAEGCKTIAFSYMGPDVTHPIYLDGTLGRAKIDLHQTSHALNLKLANFDGGAYAVVCKALVTKASVFIPGLSPYLLALYQVMKNKGTHEGCIEQMQRLFSDKLYGHSRIPLDSERLIRMDDWEMNPDTQVQVRERLQQMNASNFQQLGDYAGFKREFMQLNGFEFDQIDYSQS

SEQ ID NO: 47 - protein, 387 aa, Clostridium acetobutylicum
MIVKAKFVKGFIRDVHPYGCRREVLNQIDYCKKAIGFRGPKKVLIVGASSGFGLATRISVAFGGPEAHTIGVSYETGATDRRIGTAGWYNNIFFKEFAKKKGLVAKNFIEDAFSNETKDKVIKYIKDEFGKIDLFVYSLAAPRRKDYKTGNVYTSRIKTILGDFEGPTIDVERDEITLKKVSSASIEEIEETRKVMGGEDWQEWCEELLYEDCFSDKATTIAYSYIGSPRTYKIYREGTIGIAKKDLEDKAKLINEKLNRVIGGRAFVSVNKALVTKASAYIPTFPLYAAILYKVMKEKNIHENCIMQIERMFSEKIYSNEKIQFDDKGRLRMDDLELRKDVQDEVDRIWSNITPENFKELSDYKGYKKEFMNLNGFDLDGVDYSKD

SEQ ID NO: 48 - protein, 412 aa, Xylella fastidiosa
MDWVARSTTYNMLQLQHSQEILPLIIHPKTRGFICTTTHPVGCEYNVLEQIQSTRARGVRSNGPKKVLVIGASSGYGLATRISAAFGFGADTLGVFFEKPGTEKKPGTAGWYNAAAFDKSAKNAGLYSRSINGDAFSDEMRAKVIEIIKSEMGGHVDLVVYSLASPLRKMPSTGEIKRSVLKPIGVAHISNAIDTNKDQIIQATVEPATEQEIADTVAVMGGQDWELWINALTQADVLAPQTRTVAFSYIGTEITWPIYWHGALGKAKADLDATSRRLDARLQALGGGANVAVLKSVVTQASAAIPALPLYIAIVFKVMKEKGLHEGTIEQADRLLRERLYREDGQPAAVDEEHRLRLDDWELREDVQATCKVIWKQVTNENLFQLTDYANYKRDFLKLFGFERADVDYDAD

SEQ ID NO: 49 - protein, 388 aa, Coxiella burnetii
MIVQPKVRGFICTTAHPEGCARHVGEWINYAKQEPSLTGGPQKVLIIGASTGFGLASRIVAAFGAGAKTIGVFFERPASGKRTASPGWYNTAAFEKTALAAGLYAKSINGDAFSDEIKQQTIDLIQKDWQGGVDLVIYSIASPRRVHPRTGEIFNSVLKPIGQTYHNKTVDVMTGEVSPVSIEPATEKEIRDTEAVMGGDDWALWINALFKYNCLAEGVKTVAFTYIGPELTHAVYRNGTIGRAKLHLEKTARELDTQLESALSGQALISVNKALVTQASAAIPVVPLYISLLYKIMKEKNIHEGCIEQMWRLFKERLYSNQNIPTDSEGRIRIDDWEMREDVQAEIKRLWESINTGNVETVSDIAGYREDFYKLFGFGLNGIDYERG

SEQ ID NO: 50 - protein, 412 aa, Xylella fastidiosa
MDWVVRSITYNMLQLQHSQEILLLIIHPKTRGFICTTTHPVGCEYNVLEQIQSTRARGVRSNGPKKVVVIGASSGYGLATRISAAFGFGADTLGVFFEKPGTEKKPGTAGWYNAAAFDKSAKNAGLYSRSINGDAFSDEMRAKVIEIIKSEMGGHVDLVVYSLASPLRKMPSTGEIKRSVLKPIGVAHTSNAIDTNKDQIIQATVEPATEQEIADTVAVMGGQDWELWINALAQADVLAPQTRTVAFSYIGTEITWPIYWHGALGKAKADLDATSRRLDARLQFLGGGANVAVLKSVVTQASAAIPALPLYIAIVFKVMKEKGLHEGTIEQADRLLRERLYREDGQPAAIDEEHRLRLDDWELREDVQAACKVIWEQVTNENLFQLTDYANYKRDFLKLFGFERADVDYDAD

SEQ ID NO: 51 - protein, 388 aa, Vibrio parahaemolyticus
MRIEPLIQGVVARSAHPYGCHASIKEQIEYVKKAPKIKSGPKRVLIIGASSGFGLAARIALTFGGAEADTIGVSFERGPSEKGVGSAGWYNNIFFKQEATHAGRTAINIVGDAFSDSVRNEVIEAIETYFEGEVDLVIYSLAAGVRPKPHSDTFWRSVIKPIGESVTGASILLENDQWVETTLEPATEEEAEATIKVMGGEDWESWIDTLINTESVAQGCKTIAFSYMGPEVTHPIYLDGTLGRAKIDLHQTSHALNLKLANFDGGAYATVCKALVTKASVFIPALSPYLLALYRVMKEKGTHERCIEQMQRLFTTKLYDQPKVPVDGERLIRIDDLELDPQTQAEVSHLLEQMNTENFKECGDYQGFKDEFMKLNGFNFDDVDYSQD

SEQ ID NO: 52 - protein, 388 aa, Vibrio parahaemolyticus
MIIKPRIRGFICTTTHPVGCEANVKEQIAYTKAQGPIKNAPKRVLVVGASSGYGLSSRIAAAFGGGASTIGVFFEKEGTEKKPGTAGFYNAAAFEKLAREEGLYAKSLNGDAFSNEAKQKTIDLIKEDLGQVDMVVYSLASPVRKMPETGELIRSALKPIGETYTSTAVDTNKDVIIEASVEPATEEEIKDTVTVMGGEDWELWINALSDAGVLAEGCKTVAYSYIGTELTWPIYWDGALGKAKMDLDRAAKALNEKLGATGGSANVAVLKSVVTQASSAIPVMPLYIAMVFKKMREEGVHEGCMEQIYRMFSQRLYKEDGSAAEVDDMNRLRLDDWELREDIQQHCRELWPQITTENLKELTDYVEYKEEFLKLFGFGVEGVDYEAD

SEQ ID NO: 53 - protein, 415 aa, Burkholderia fungorum
MGAAQAANRRGAPATIEAHYNVANSDKRCFMIIKPRVRGFICVSTHPTGCEANVREQIEYVKARGPIANGPKKVLVIGASTGYGLAARISAAFGSDAATLGVFFERAGSETKAGTAGWYNTAAFEKFATEQGLYATSINGDAFSDAVKQRTIEVIKRDLGQVDLVVYSLAAPKRTHPKSGEVFSSTLKPVGQAVNLRGIDTDKEVIRETVLEPATQDEIDHTVAVMGGEDWQMWIDALLEAGVLADGAKTTAFTYLGEKITHDIYWNGSIGAAKKDLDQKVLGIREKLAAKGGDARVSVLKAVVTQASSAIPMMPLYLSLLFKVMKEKGTHEGCIEQVYGLYKDSLYGAAPHLDDEGRLRADYKELDPEVQDRVQTLWSQVTNDNIYELTDFSGYKTDFLRLFGFEIAGVDYEAD

SEQ ID NO: 54 - protein, 385 aa, Microbulbifer degradans
MIIKPKVRGFICTNAHPEGCAANVREQIDFVKSQGEIADGPKKVLVIGASTGYGLASRITAAFGCGAATLGVFFEKPGTERKTGSAGFYNSVGFHAEAEKAGLYAKSINGDAFSDEIKAKAIDVIKRDLGKVDLVVYSLASPRRTDPKTGELYSSVLKPIGQSYTAKNLNTDTLKIADMTIEAANEEEIANTVKVMGGEDWELWVEALKEADVLAENAKTVAYTYIGDKLTWPIYGKATIGRAKEDLDRAATAINQKLADLGGSANVAVLKALVTQSSSAIPIMPLYISILYKVMKEEGTHEGCIEQLYRLFTEGVYTDTPRLDDANRFRMDEKELAAELQAKVEAIWPNVTEENLFEETDYKGYNAEFLKLFGFGVDGIDYEKD

SEQ ID NO: 55 - protein, 385 aa, Cytophaga hutchinsonii
MIIEPKMRGFICLTSHPTGCEQNVINQINYVKSKGVINGPKKVLVIGASTGFGLASRITSAFGSNAATIGVFFEKPAQEGKPGSPGWYNTVAFQNEAKKAGIYAKSINGDAFSTEVKQKTIDLIKADLGQVDLVIYSLASPVRTNPVTGVTHRSVLKPIGGAFSNKTVDFHTGNVSTVTIEPANEEDVTNTVAVMGGEDWGMWMDAMLEAGVLAEGATTVAYSYIGPALTEAVYRKGTIGRAKDHLEASAATITDKLKSVKGKAYVSVNKALVTQASSAIPVIPLYISLLYKVMKAEGIHEGCIEQIQRLYADRLYTGKAIPTDEQGRIRIDDWEMREDVQANVAALWEQVTSENVSDISDLKGYKNDFLNLFGFAVNKVDYLAD